(19) United States
(12) Patent Application Publication
(10) Pub. No.: US 2014/0335136 A1
(43) Pub. Date: Nov. 13, 2014

(54) COMPOSITIONS FOR MINIMIZING SKIN IMPERFECTIONS

(71) Applicant: L'OREAL, Paris (FR)

(72) Inventors: Patricia BRIEVA, Manalapan, NJ (US); Mark ZAW, Denville, NJ (US); Yinli WANG, Ramsey, NJ (US); Donna McCANN, Oxford, NJ (US); Anthony POTIN, Hoboken, NJ (US); Kelly LOCKWOOD, Waymart, PA (US); Guillaume CASSIN, Villebon-sur-Yvette (FR)

(21) Appl. No.: 13/890,829

(22) Filed: May 9, 2013

(51) Int. Cl. A61K 8/58; A61K 8/19; A61Q 17/00; A61Q 19/00; A61Q 1/02; A61K 8/25; A61K 8/36

(52) U.S. Cl. CPC A61K 8/585; A61K 8/25; A61K 8/19; A61K 8/361; A61Q 19/00; A61Q 1/02; A61Q 17/00
USPC 424/401; 424/63; 424/59; 514/63

(57) ABSTRACT

Disclosed herein are compositions comprising, in a cosmetically acceptable medium, a silicone material comprising concave and/or annular particles; hydrophobic silica aerogel particles; fillers chosen from platelet type fillers and silica particles other than hydrophobic silica aerogel particles; a fatty substance chosen from fatty alcohols and fatty acids; an emulsifying agent and a thickening agent, wherein the silica material, hydrophobic silica aerogel particles and fillers are present in a total amount of greater than 4% by weight, based on the total weight of the composition. Also disclosed herein is a process for reducing the visibility of imperfections on skin, comprising applying the composition onto the skin.

### FIELD OF THE INVENTION

The present invention relates to a composition containing an association of particulate materials, a fatty substance, an emulsifying agent, a thickening agent and solvent comprising water. This composition allows for decreasing the visibility of pores once applied on the skin and for achieving an even skin tone, while reducing the appearance of skin discolorations.

### BACKGROUND

Imperfections of the skin are visible because of the contrast between bright areas such as skin ridges and dark areas such as skin pores. Imperfections of the skin may also result from uneven pigmentation of the skin, resulting in uneven skin tone or bright and dark areas on the skin. Light scattering can decrease this brightness gap and may be achieved by matte and haze/blurring effects or optical effects. Thus, particulate and filler materials, such as pigments and silica-based compounds with mattifying and/or light scattering effects can be employed in cosmetic and skin care compositions in order to obtain desirable optical effects.

In formulating cosmetic and skin care compositions which contain particulate and filler materials, the types, amounts and/or physical/functional properties of these materials, particularly the strength of their light scattering properties and their oil absorption capacities, may impact the efficacies of said compositions. For example, some of these materials tend to accumulate inside the pores of the skin and increase pore visibility which is highly undesirable. The use of these materials also sometimes result into a composition that imparts a visible coating or layer on the skin such as a whitish coating that is not desirable, particularly when the composition is used as a skin care product or as a base composition before making up the skin with colored cosmetics. Another challenge in formulating with particle and filler materials is the selection of other ingredients such that the final composition is stable and possesses other cosmetic attributes, for example, good texture and able to impart a soft, smooth feel to the skin.

Thus, there remains a need for having cosmetic or skin care compositions containing particulate and filler materials, wherein said compositions have improved properties of minimizing skin imperfections such as visible pores and uneven skin tone or color, while at the same, having other desirable cosmetic properties such as good texture and a translucent/transparent or natural appearance on the skin.

So far, cosmetic products employed to hide pores and to even skin tone include silicon elastomers and/or spherical fillers and/or plate fillers such as barium sulfate, composite materials, small pearls. US2009081316 from Momentive describes the association of silicon elastomer and boron nitride. US2005163730 from Unilever describes the association of silicon elastomer, ZnO nanopigment and plate type filler such as TiO₂ coated mica or bismuth oxychloride. WO2012066097 from L'Oreal describes the association of a white pigment such as TiO₂, ZnO, barium salts and bismuth oxychloride and a filler such as silica-based materials.

It has now been surprisingly discovered that by incorporating: (1) a silicone material comprising concave and/or annular particles; (2) a hydrophobic silica aerogel particle; (3) at least two fillers chosen from platelet type fillers and silica particles other than the hydrophobic silica aerogel particle; (4) a fatty substance chosen from fatty alcohols and fatty acids; (5) an emulsifying agent; and (6) a thickening agent into a composition, said composition, when applied onto skin, allows for a reduction in the visibility of pores and evenness of skin tone or color of skin.

### SUMMARY OF THE INVENTION

The present invention relates to a composition containing, in a cosmetically acceptable medium:
a) at least one silicone material comprising concave and/or annular particles;
b) at least one hydrophobic silica aerogel particle;
c) at least two fillers selected from the group comprising platelet type fillers and silica particles other than (b);
d) at least one fatty substance selected from the group comprising fatty alcohols and fatty acids;
e) at least one emulsifying agent selected from the group comprising oxyethylenated and/or oxypropylenated ethers of glycerol, oxyethylenated and/or oxypropylenated ethers of fatty alcohols, fatty acid esters of polyethylene glycol, fatty acid esters of oxyethylenated and/or oxypropylenated glyceryl ethers, fatty acid esters of polyols and mixtures thereof; and
f) at least one thickening agent;
   wherein (a), (b) and (c) are present in a total amount of greater than 4% by weight, based on the total weight of the composition.

The present invention also relates to a process of reducing the visibility of imperfections and/or imparting a homogenizing effect on skin, comprising applying the above-described composition onto the skin.

The invention also concerns a process comprising a step of applying at least one layer of the composition according to the invention onto the skin, in particular, the skin of the face.

In certain embodiments, the composition of the invention is a skin care composition.

In other embodiments, the composition of the invention is a make-up composition.

In a particular embodiment, the composition of the invention is a base or a primer, in particular a skin care or a make-up base or primer. Thus, in certain embodiments, the composition is applied as a base or a primer under a skin care product or a make-up product.

The composition according to the invention is particularly intended to decrease the visibility of skin imperfections, in particular the pores or uneven skin tone or color or skin discolorations or irregular marks on the skin.

Preferably, the composition of the present invention is in the form of one of the following: a skin care product, a make-up product (for example, a liquid foundation), a suncare product, a concealer product, a skin care or make-up base or primer composition. Preferably, the composition is an emulsion, such as for example, an oil in water emulsion.

According to the present invention, the process of reducing the visibility of skin imperfections is also to be understood as a process of minimizing skin imperfections or minimizing the appearance of skin imperfections. Said process can result in pore minimization and/or a reduction in color variability.

It is to be understood that both the foregoing general description and the following detailed description are exemplary and explanatory only, and are not restrictive of the invention.

### BRIEF DESCRIPTION OF THE FIGURES

FIG. 1 shows examples of contrast cards that may be used to determine the colorimetric effects of the compositions of the invention.

FIG. 2 shows the various areas on a contrast card from FIG. 1 which can correspond to average colors observed on a panel of individuals having dark skin with a dark complexion with respect to various regions of the face.

FIG. 3 shows the efficacy of minimizing skin imperfections as imparted by various compositions, including the compositions of the invention, once applied onto skin.

FIG. 4 shows images of bare skin and skin onto which the composition of the invention was applied.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 5% to 10% of the indicated number.

The term “skin” means the skin on the face or the lips or the skin around the eyes or the skin on the neck and chest areas or other parts of the body.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100 degrees C.

“Non-volatile”, as used herein, means having a flash point of greater than about 100 degree C.

The term “filler” as used herein should be understood to mean colorless or white solid particles either of any shape or of a shape as specified herein which are in a form that is insoluble and dispersed in the medium of the composition.

The term “pilling” as used herein refers to a composition or material rubbing off the skin during product application, thereby leaving a residue in clumps of small balls or the like, resembling pills, on the skin (“pilling”).

According to the present invention, a homogenizing effect on the skin is to be understood as obtaining an even skin tone or color and/or producing a blurring/haze or soft focus effect on the skin such that the appearance of discolorations, blemishes, pores, fine lines or wrinkles is minimized by applying the compositions of the present invention. At the same time, the homogenizing effect imparted by the compositions of the present invention may be accompanied by a transparency effect wherein the compositions, once applied onto skin, do not produce an undesirable whitish sheen or film on the skin but are transparent/translucent such that the skin has a natural appearance.

A homogenizing effect according to the present invention can also be described in terms of the homogenizing power of the composition wherein the homogenizing power is measured in accordance with a protocol for measuring or evaluating the efficacy of the compositions with respect to minimizing the visibility of skin imperfections described below one said composition is applied onto skin. Homogenizing power may also be described as covering power.

The compositions and methods of the present invention can comprise, consist of, or consist essentially of the essential elements and limitations of the invention described herein, as well as any additional or optional ingredients, components, or limitations described herein or otherwise useful.

It was surprisingly and unexpectedly discovered that the compositions of the present invention, when applied onto skin, formed a film or coating on the skin that reduced the visibility of skin imperfections, such as pores, lines, wrinkles, unevenness of skin tone or color or skin discolorations. The compositions of the present invention can be characterized as having a homogenizing power such that when applied onto skin, the visibility of said skin imperfections was significantly reduced.

It was also surprisingly and unexpectedly discovered that when the compositions of the present invention were in the form of a skin care or a base or primer, the skin's natural complexion was unchanged, resulting in a bare skin or natural look appearance, while making it possible to be free of the use of foundation. This means that the compositions formed a coating or film that was sufficiently transparent such that said film or coating was not visibly noticeable.

Furthermore, it was surprisingly and unexpectedly discovered that the combination of fatty substances chosen from fatty acids and fatty alcohols, an emulsifying agent and a thickening agent in a cosmetically acceptable medium and in association with a silicone material comprising concave and/or annular particles, hydrophobic silica aerogel particles, at least two fillers chosen from platelet type fillers and silica particles other than the hydrophobic silica aerogel particles resulted in a composition having excellent homogenizing whereby said composition significantly reduced the visibility of skin imperfections.

It was also found that the compositions of the present invention are stable and have a novel texture wherein said compositions are smooth and/or creamy, are easy to apply and spread on the skin, are non-pilling on the skin, and impart a smooth and soft feel to the skin.

Silicone Material Comprising Concave and/or Annular Particles

The concave and/or annular particles present in the composition according to the invention are silicone particles, in particular particles formed of portions of hollow spheres composed of a silicone material.

The particles preferably have a mean diameter of less than or equal to 10 μm, in particular ranging from 0.1 μm to 8 μm, preferably from 0.2 to 7 μm, more preferably ranging from 0.5 to 6 μm and preferably again ranging from 0.5 to 4 μm.

The term “mean diameter” is understood to mean the greatest dimension of the particle.

Advantageously, these particles have a density of greater than 1.

The portions of hollow spheres used in the composition according to the invention include those having the shape of truncated hollow spheres exhibiting a single orifice communicating with their central cavity and having a transverse cross section with the shape of a horseshoe or arch.

The silicone material is a crosslinked polysiloxane with a three-dimensional structure; it preferably comprises, indeed may even be entirely composed of, units of formula (I) SiO₂ and of formula (II) R¹SiO_{1.5} in which R¹ denotes an organic group having a carbon atom directly connected to the silicon atom.

The organic group R¹ can be a reactive organic group; R¹ can more particularly be an epoxy group, a (meth)acryloyloxy group, an alkenyl group, a mercaptoalkyl, aminoalkyl or haloalkyl group, a glyceroxy group, a ureido group or a cyano group and preferably an epoxy group, a (meth)acryloyloxy group, an alkenyl group or a mercaptoalkyl or aminoalkyl group. These groups generally comprise from 2 to 6 carbon atoms, in particular from 2 to 4 carbon atoms.

The organic group R¹ can also be an unreactive organic group; R¹ can then more particularly be a C1-C4 alkyl group, in particular a methyl, ethyl, propyl or butyl group, or a phenyl group and preferably a methyl group.

Mention may be made, as epoxy group, of a 2-glycidoxyethyl group, a 3-glycidoxypropyl group or a 2-(3,4-epoxycyclohexyl)propyl group.

Mention may be made, as (meth)acryloyloxy group, of a 3-methacryloyloxypropyl group or a 3-acryloyloxypropyl group.

Mention may be made, as alkenyl group, of the vinyl, allyl or isopropenyl groups.

Mention may be made, as mercaptoalkyl group, of the mercaptopropyl or mercaptoethyl groups.

Mention may be made, as aminoalkyl group, of a 3-[(2-aminoethyl)amino]propyl group, a 3-aminopropyl group or an N,N-dimethylaminopropyl group.

Mention may be made, as haloalkyl group, of a 3-chloropropyl group or a trifluoropropyl group.

Mention may be made, as glyceroxy group, of a 3-glyceroxypropyl group or a 2-glyceroxyethyl group.

Mention may be made, as ureido group, of a 2-ureidoethyl group.

Mention may be made, as cyano group, of the cyanopropyl or cyanoethyl groups.

Preferably, in the unit of formula (II), R¹ denotes a methyl group.

Advantageously, the silicone material comprises the units (I) and (II) according to a unit (I)/unit (II) molar ratio ranging from 30/70 to 50/50, preferably ranging from 35/65 to 45/55.

The particles of silicone material can in particular be capable of being obtained according to a process comprising:

a) the introduction into an aqueous medium, in the presence of at least one hydrolysis catalyst and optionally of at least one surfactant, of a compound (III) of formula SiX₄ and of a compound (IV) of formula RSiY₃, where X and Y denote, independently of one another, a C1-C4 alkoxy group, an alkoxyethoxy group including a C1-C4 alkoxy group, a C2-C4 acyloxy group, an N,N-dialkylamino group including C1-C4 alkyl groups, a hydroxyl group, a halogen atom or a hydrogen atom and R denotes an organic group comprising a carbon atom connected directly to the silicon atom; and

(b) the operation in which the mixture resulting from stage (a) is brought into contact with an aqueous solution including at least one polymerization catalyst and optionally at least one surfactant, at a temperature of between 30 and 85 degrees C., for at least two hours.

Stage (a) can be characterized as a hydrolysis reaction and stage (b) can be characterized as a condensation reaction.

In stage (a), the molar ratio of the compound (III) to the compound (IV) usually ranges from 30/70 to 50/50, advantageously from 35/65 to 45/55, and is preferably 40/60. The ratio by weight of the water to the total of the compounds (III) and (IV) preferably ranges from 10/90 to 70/30. The order of introduction of the compounds (III) and (IV) generally depends on their rate of hydrolysis. The temperature of the hydrolysis reaction generally ranges from 0 to 40 degrees C. and usually does not exceed 30 degrees C. in order to prevent premature condensation of the compounds.

For the X and Y groups of the compounds (III) and (IV): mention may be made, as C1-C4 alkoxy group, of the methoxy or ethoxy groups; mention may be made, as alkoxyethoxy group including a C1-C4 alkoxy group, of the methoxyethoxy or butoxyethoxy groups; mention may be made, as C2-4 acyloxy group, of the acetoxy or propionyloxy groups; mention may be made, as N,N-dialkylamino group including C1-C4 alkyl groups, of the dimethylamino or diethylamino groups; mention may be made, as halogen atom, of the chlorine or bromine atoms.

Mention may be made, as compounds of formula (III), of tetramethoxysilane, tetraethoxysilane, tetrabutoxysilane, trimethoxyethoxysilane, tributoxyethoxysilane, tetraacetoxysilane, tetrapropioxysilane, tetra(dimethylamino)silane, tetra(diethylamino)silane, silanetetraol, chlorosilanetriol, dichlorodisilanol, tetrachlorosilane or chlorotrihydrosilane. Preferably, the compound of formula (III) is chosen from tetramethoxysilane, tetraethoxysilane, tetrabutoxysilane and their mixtures.

The compound of formula (III) results, after the polymerization reaction, in the formation of the units of formula (I).

The compound of formula (IV) results, after the polymerization reaction, in the formation of the units of formula (II).

The R group in the compound of formula (IV) has the meaning as described for the R 1 group for the compound of formula (II).

Mention may be made, as examples of compounds of formula (IV) comprising an unreactive organic group R, of methyltrimethoxysilane, ethyltriethoxysilane, propyltributoxysilane, butyltributoxysilane, phenyltrimethoxyethoxysilane, methyltributoxyethoxysilane, methyltriacetoxysilane, methyltripropioxysilane, methyltri(dimethylamino)silane, methyltri(diethylamino)silane, methylsilanetriol, methylchlorodisilanol, methyltrichlorosilane or methyltrihydrosilane.

Mention may be made, as examples of compounds of formula (IV) comprising a reactive organic group R, of:

silanes having an epoxy group, such as (3-glycidoxypropyl)trimethoxysilane, (3-glycidoxypropyl)triethoxysilane, [2-(3,4-epoxycyclohexyl)ethyl]trimethoxysilane, (3-glycidoxypropyl)methyldimethoxysilane, (2-glycidoxyethyl)methyldimethoxysilane, (3-glycidoxypropyl)dimethylmethoxysilane or (2-glycidoxyethyl)dimethylmethoxysilane;

silanes having a (meth)acryloyloxy group, such as (3-methacryloyloxypropyl)trimethoxysilane or (3-acryloyloxypropyl)trimethoxysilane;

silanes having an alkenyl group, such as vinyltrimethoxysilane, allyltrimethoxysilane or isopropenyltrimethoxysilane;

silanes having a mercapto group, such as mercaptopropyltrimethoxysilane or mercaptoethyltrimethoxysilane;

silanes having an aminoalkyl group, such as (3-aminopropyl)trimethoxysilane, (3-[(2-aminoethyl)-amino]propyl)trimethoxysilane, (N,N-dimethylaminopropyl)trimethoxysilane or (N,N-dimethylaminoethyl)trimethoxysilane;

silanes having a haloalkyl group, such as (3-chloropropyl)trimethoxysilane or trifluoropropyltrimethoxysilane;

silanes having a glyceroxy group, such as (3-glyceroxypropyl)trimethoxysilane or di-(3-glyceroxypropyl)dimethoxysilane;

silanes having a ureido group, such as (3-ureidopropyl)trimethoxysilane, (3-ureidopropyl)-methyldimethoxysilane or (3-ureidopropyl)dimethylmethoxysilane;

silanes having a cyano group, such as cyanopropyltrimethoxysilane, cyanopropylmethyldimethoxysilane or cyanopropyldimethylmethoxysilane.

Preferably, the compound of formula (IV) comprising a reactive organic group R is chosen from silanes having an epoxy group, silanes having a (meth)acryloyloxy group, silanes having an alkenyl group, silanes having a mercapto group or silanes having an aminoalkyl group.

Examples of compounds (III) and (IV) which are preferred for the implementation of this invention are respectively tetraethoxysilane and methyltrimethoxysilane.

Use may independently be made, as hydrolysis and polymerization catalysts, of basic catalysts, such as sodium hydroxide, potassium hydroxide, sodium carbonate, sodium hydrogencarbonate, ammonia or amines, such as trimethylamine, triethylamine or tetramethylammonium hydroxide, or acidic catalysts, such as organic acids, for example citric acid, acetic acid, methanesulphonic acid, p-toluenesulphonic acid, dodecylbenzenesulphonic acid or dodecylsulphonic acid, or inorganic acids, such as hydrochloric acid, sulphuric acid or phosphoric acid.

When it is present, the surfactant used is preferably a nonionic or anionic surfactant or a mixture of the two. Sodium dodecylbenzenesulphonate can be used as anionic surfactant. The end of the hydrolysis is marked by the disappearance of the products (III) and (IV), which are insoluble in water, and the production of a homogeneous liquid layer.

The condensation stage (b) can use the same catalyst as the hydrolysis stage or another catalyst chosen from those mentioned above.

On conclusion of this process, a suspension in water of fine organosilicone particles is obtained, which particles can optionally be separated subsequently from their medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

The shape of the portions of hollow spheres obtained according to the above process and their dimensions will depend in particular on the method used to bring the products into contact in stage (b).

A somewhat basic pH and introduction under cold conditions of the polymerization catalyst into the mixture resulting from stage (a) will result in portions of hollow spheres with the shape of round-bottomed “bowls”, whereas a somewhat acidic pH and dropwise introduction of the mixture resulting from stage (a) into the hot polymerization catalyst will result in portions of hollow spheres having a transverse cross section with the shape of a “horseshoe”.

According to a preferred embodiment of the invention, portions of hollow spheres with the shape of “bowls” are used. These can be obtained as described in Application JPA-2003-128788.

Portions of hollow spheres with the shape of a horseshoe are described in Application JP-A-2000-191789.

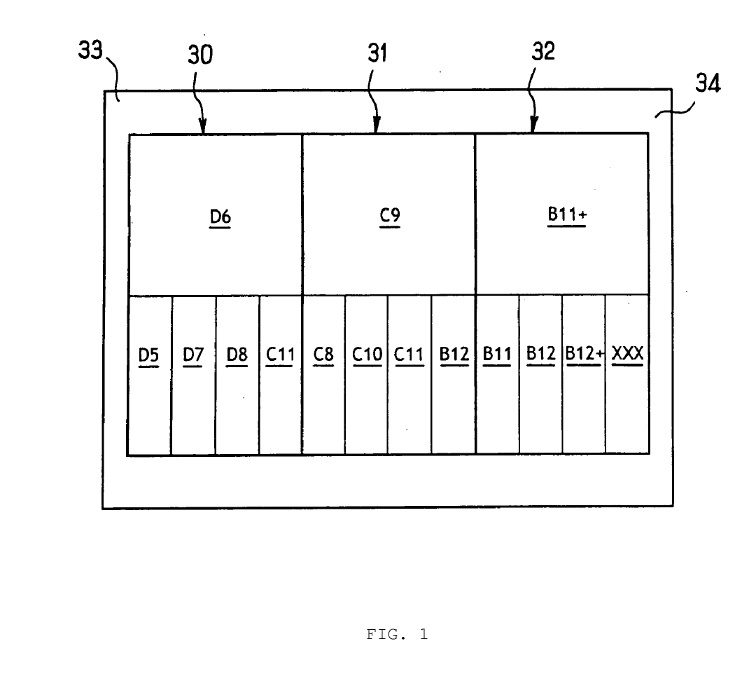

A concave particle in the form of portions of spheres with the shape of a bowl is illustrated in transverse cross section in FIG. 1 of U.S. patent application, 2008/0108535A1, and described in said U.S. patent application.

Mention may be made, as concave particles in the form of portions of spheres which can be used according to the invention, of, for example:

particles composed of the crosslinked organosilicone TAK-110 (crosslinked methylsilanol/silicate polymer) from Takemoto Oil and Fat, with the shape of a bowl, with a width of 2.5 μm, a height of 1.2 μm and a thickness of 150 nm (particles sold under the name NLK-506 by Takemoto Oil and Fat);

particles composed of the crosslinked organosilicone TAK-110 (crosslinked methylsilanol/silicate polymer) from Takemoto Oil and Fat, with the shape of a bowl, with a width of 0.8 μm, a height of 0.4 μm and a thickness of 130 nm (particles sold under the name NLK-515 by Takemoto Oil and Fat);

particles composed of the crosslinked organosilicone TAK-110 (crosslinked methylsilanol/silicate polymer) from Takemoto Oil and Fat, with the shape of a bowl, with a width of 7 μm, a height of 3.5 μm and a thickness of 200 nm (particles sold under the name NLK-510 by Takemoto Oil and Fat).

The width of the crosslinked organosilicone TAK materials above correspond to the diameter of the particles.

These particles have the INCI name: Methylsilanol/silicate crosspolymer and may also be known under the tradename Diakalyte®.

Advantageously, the concave silicone particles have a mean diameter of less than or equal to 5 μm, in particular ranging from 0.1 μm to 5 μm, preferably ranging from 0.2 to 5 μm, more preferably ranging from 0.5 to 4 μm and preferably again ranging from 0.5 to 3 μm.

These particles make possible, in addition to the reduction, indeed even the elimination, of the sticky feel, the optimization of the properties of slip, of spreading and of comfort of the composition according to the invention.

The silicone particles of annular shape are preferably chosen from those described and synthesized in Patent Application US-A-2006/0089478. They exhibit a mean external diameter of 0.05 to 15 μm and a mean internal diameter of 0.01 to 10 μm, the difference between the mean external diameter and the mean internal diameter being from 0.04 to 5 μm.

They exhibit a polysiloxane network comprising siloxane units of formulas (1), (2), (3), (4), (5) and (6):

S¹O_{4/2} (1)

Si(OH)_{3/2} (2)

R₂SiO_{3/2} (3)

R₂SiO_{3/2} (4)

R₃(SiOH)_{2/2} (5)

R₄(SiOH)_{2/2} (6)

in which:

R₁ and R₃ denote unreactive hydrocarbon groups, in particular alkyl, cycloalkyl, aryl, alkylaryl or aralkyl groups, preferably C1-C3 alkyl groups, in particular methyl, ethyl or propyl groups and preferably a methyl group,

R₂ and R₄ each denote a hydrocarbon group chosen from the acryloyloxy, methacryloyloxy, vinyl or mercapto groups; the siloxane units of formula (1)/siloxane units of formulae (2), (3), (4), (5) and (6) molar ratio being from 20/80 to 50/50; the siloxane units of formulae (2), (3) and (4)/siloxane units of formulae (5) and (6) molar ratio being from 50/50 to 75/25; the siloxane units of formulae (3) and (5)/siloxane units of formulae (4) and (6) molar ratio being from 20/80 to 60/40.

Mention may be made, as acryloyloxy group, of a 2-methacryloyloxyethyl group or a 3-acryloyloxypropyl group.

Mention may be made, as (meth)acryloyloxy group, of a 3-methacryloyloxypropyl group or a 3-acryloyloxypropyl group.

Mention may be made, as mercaptoalkyl group, of a mercaptopropyl or mercaptoethyl group.

Mention may be made, as vinyl group, of the allyl, isopropenyl or 2-methylallyl groups.

According to the present invention, a preferred silicone material comprising concave and/or annular particles of the present invention is methylsilanol/silicate crosspolymer, known under the tradename NLK-506 and commercially available from the company, Takemoto Oil and Fat. Other preferred silicone materials comprising concave and/or annular particles of the methylsilanol/silicate crosspolymer type are known under the tradenames NLK-515 and NLK-510 (Takemoto Oil and Fat).

The at least one silicone material comprising concave and/or annular particles in the composition according to the invention may be present in the composition in an amount ranging from 0.1 to 15% by weight, preferably ranging from about 0.5 about to 5% by weight, more preferably ranging from about 0.5 to about 3% by weight, and even more preferably ranging from about to about 2% by weight, based on the total weight of the composition, including all ranges and sub ranges there between. For example, the silicone material comprising concave and/or annular particles for use in the composition of the present invention may be present at 0.5% by weight, or at 1% by weight, or at 1.25% by weight, or at 1.5% by weight, or at 1.75% by weight, or at 2% weight, all weights being based on the total weight of the composition.

### Hydrophobic Silica Aerogel Particles

Silica aerogels are porous materials obtained by replacing (by drying) the liquid component of a silica gel with air.

They are generally synthesized via a sol-gel process in liquid medium and then dried, usually by extraction of a supercritical fluid, the one most commonly used being supercritical CO2. This type of drying makes it possible to avoid shrinkage of the pores and of the material. The sol-gel process and the various drying processes are described in detail in Brinker C J., and Scherer G. W., Sol-Gel Science: New York: Academic Press, 1990.

The hydrophobic silica aerogel particles that may be used in the present invention have a specific surface area per unit of mass (SM) ranging from 500 to 1500 m2/g, preferably from 600 to 1200 m2/g and better still from 600 to 800 m2/g, and a size expressed as the mean volume diameter (D[0.5]), ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles that may be used in the present invention have a size expressed as the mean volume diameter (D[0.5]) ranging from 1 to 30 μm, preferably from 5 to 25 μm, better still from 5 to 20 μm and even better still from 5 to 15 μm.

The specific surface area per unit of mass may be determined via the BET (Brunauer-Emmett-Teller) nitrogen absorption method described in the Journal of the American Chemical Society, vol. 60, page 309, February 1938 and corresponding to the international standard ISO 5794/1 (appendix D). The BET specific surface area corresponds to the total specific surface area of the particles under consideration.

The size of the hydrophobic silica aerogel particles may be measured by static light scattering using a commercial granulometer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles,” Chapters 9 and 10, Wiley, New York, 1957.

According to one advantageous embodiment, the hydrophobic silica aerogel particles used in the present invention have a specific surface area per unit of mass (SM) ranging from 600 to 800 m2/g and a size expressed as the mean volume diameter (D[0.5]) ranging from 5 to 20 μm and better still from 5 to 15 μm.

The hydrophobic silica aerogel particles used in the present invention may advantageously have a tamped density p ranging from 0.04 g/cm3 to 0.10 g/cm3 and preferably from 0.05 g/cm3 to 0.08 g/cm3.

In the context of the present invention, this density, known as the tamped density, may be assessed according to the following protocol:

40 g of powder are poured into a measuring cylinder; the measuring cylinder is then placed on a Stay 2003 machine from Stampf Volumeter; the measuring cylinder is then subjected to a series of 2500 packing motions (this operation is repeated until the difference in volume between two consecutive tests is less than 2%); the final volume Vf of packed powder is then measured directly on the measuring cylinder. The tamped density is determined by the ratio m/Vf, in this instance 40/Vf (Vf being expressed in cm³ and m in g).

According to one embodiment, the hydrophobic silica aerogel particles that may be used in the present invention have a specific surface area per unit of volume SV ranging from 5 to 60 m2/cm3, preferably from 10 to 50 m2/cm3 and better still from 15 to 40 m2/cm3.

The specific surface area per unit of volume is given by the relationship: SV=SM. ρ; where ρ is the tamped density expressed in g/cm3 and SM is the specific surface area per unit of mass expressed in m2/g, as defined above.

Preferably, the hydrophobic silica aerogel particles according to the invention have an oil-absorbing capacity, measured at the wet point, ranging from 5 to 18 ml/g, preferably from 6 to 15 ml/g and better still from 8 to 12 ml/g.

The oil-absorbing capacity measured at the wet point, noted Wp, corresponds to the amount of water that needs to be added to 100 g of particle in order to obtain a homogeneous paste.

It is measured according to the wet point method or the method for determining the oil uptake of a powder described in standard NF T 30-022. It corresponds to the amount of oil adsorbed onto the available surface of the powder and/or absorbed by the powder by measuring the wet point, described below:

An amount m=2 g of powder is placed on a glass plate, and the oil (isononyl isononanoate) is then added dropwise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted.

The oil uptake corresponds to the ratio Vs/m.

The hydrophobic silica aerogel particles that may be used according to the present invention are preferably of silylated silica type (INCI name: silica silylate).

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

As regards the preparation of hydrophobic silica aerogels particles that have been surface-modified by silylation, reference may be made to document U.S. Pat. No. 7,470,725.

Use will be made in particular of hydrophobic silica aerogels particles surface-modified with trimethylsilyl groups with INCI name Silica silylate.

As hydrophobic silica aerogel particles that may be used in the invention, examples that may be mentioned include the aerogel sold under the name VM-2260 or VM-2270 (INCI name: Silica silylate), by the company Dow Corning, the particles of which have a mean size of about 1000 microns and a specific surface area per unit of mass ranging from 600 to 800 m2/g.

Mention may also be made of the aerogels sold by the company Cabot under the references Aerogel® TLD 201, Aerogel® OGD 201, Aerogel® TLD 203, and ENOVA AEROGEL® MT 1100.

According to the present invention, a preferred hydrophobic silica aerogel particle is the aerogel sold under the name VM-2270 (INCI name: Silica silylate, 98% active), by the company Dow Corning, the particles of which have a mean size ranging from 5-15 microns and a specific surface area per unit of mass ranging from 600 to 800 m2/g (oil uptake equal to 1080 ml/100 g).

Advantageously, the hollow particles in accordance with the invention are at least partly formed from hydrophobic silica aerogel particles, preferably those with a specific surface area per unit of mass (SM) ranging from 500 to 1500 m2/g and preferably from 600 to 1200 m2/g, and a size expressed as the mean volume diameter (D[0.5]), ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

The use of the hollow particles according to the invention, in particular of hydrophobic silica aerogel particles, also advantageously makes it possible to improve the remanence of the cosmetic properties afforded by the composition on keratin materials, in particular the skin.

The hydrophobic silica aerogel particle for use in the composition of the present invention may be present in the composition in an amount ranging from ranging from about 0.1 to about 15% by weight, preferably ranging from about 0.5 to about 5% by weight, more preferably ranging from about 0.5 to about 3% by weight, and even more preferably ranging from about 1 to about 2% by weight, based on the total weight of the composition, including all ranges and sub ranges there between. For example, the hydrophobic silica aerogel particle for use in the composition of the present invention may be present at 0.5% by weight, or at 1% by weight, or at 1.25% by weight, or at 1.5% by weight, or at 1.75% by weight, or at 1.96% by weight or at 2% weight, all weights being based on the total weight of the composition.

### Fillers

The at least two fillers employed in the compositions of the present invention are chosen from platelet type fillers and silica particles other than the above-described hydrophobic silica aerogel particles.

In preferred embodiments of the present invention, the plate type filler has a high refractive index (RI>1.6) and a particle size from between about 1 μm and about 15 μm.

In particular, the plate type filler has refractive index ranging from between 1.6 and 2.2 and a particle size ranging from between about 1 μm and about 15 μm.

The particle size of the fillers of the present invention may be expressed as the mean volume diameter (D[0.5]).

In particular, the said plate type filler is chosen from boron nitride, barium sulfate, bismuth oxychloride, alumina and composite powders based on titanium oxide and substrate like talc, mica, barium sulfate, boron nitride, bismuth oxychloride, alumina, and mixtures thereof.

In a particular embodiment, the plate type filler is a boron nitride.

In a preferred embodiment, the plate type filler is a boron nitride having a particle size between 1 μm and 10 μm, and in particular between 1 and 6 μm.

As examples of commercial products of boron nitride, we may use the following products: SHP series from Mizushima Ferroalloy Co, LTD (mean or average particle size ranging from about 3 to about 18 μm), PUHP3008 from Saint Gobains Ceramics (mean particle size 6 μm), the PUHP1030L from Saint Gobain Ceramics (mean particle size 3 μm), the Softouch BN CC6058 powder from Momentive Performance Materials (mean particle size 5-15 μm), or mixtures thereof.

In another preferred embodiment, the plate type filler is bismuth oxychloride, which is commercially available under the tradename of Timiron® Liquid Silver from the company, Merck or under the tradename of Biron® Liquid Silver from the company, EMD Chemicals, Inc.

In preferred embodiments of the present invention, the filler may be chosen from silica particles other than the hydrophobic silica aerogel particles can be natural and non-treated. Suitable examples are those known under the trade names SILLITIN N85, SILLITIN N87, SILLITIN N82, SILLITIN V85 and SILLITIN V88, commercially available from the company Hoffmann Mineral.

The silica particles other than the hydrophobic silica aerogel particles can also be pyrogenic silica. The pyrogenic silica may comprise hydrophilic silica particles having silanol groups on their surface.

Pyrogenic silica may also be surface-hydrophobicized pyrogenic silica. It is possible in effect to modify chemically the surface of the silica, by a chemical reaction which brings about a decrease in the number of silanol groups present on the surface of the silica. In particular, it is possible to substitute the silanol groups with hydrophobic groups: in that case a hydrophobic silica is obtained. The hydrophobic groups may be:
trimethylsiloxyl groups, obtained in particular by treating pyrogenic silica in the presence of hexamethyldisilazane. Silicas treated in this way may also be known as “silica silylate” according to the CTFA (6th edition, 1995).
dimethylsilyloxyl or polydimethylsiloxane groups, obtained in particular by treating pyrogenic silica in the presence of polydimethylsiloxane or dimethydichlorosilane. Silicas treated in this way may also be known as “silica dimethyl silylate” according to the CTFA (6^{th} edition, 1995).

In particularly preferred embodiments of the present invention, the silica particles other than the hydrophobic silica aerogel particles comprise silicas powders that include:
porous silica microspheres, especially those sold under the names Sunsphere® H53 and Sunsphere® H33 by the company Asahi Glass; MSS-500-3H by the company Kobo;
polydimethylsiloxane-coated amorphous silica microspheres, especially those sold under the name SA Sunsphere® H33 by the company Asahi Glass;
amorphous hollow silica particles, especially those sold under the name Silica Shells by the company Kobo; and
precipitated silica powders surface-treated with a mineral wax, such as precipitated silica treated with a polyethylene wax, and especially those sold under the name Acematt® OK 412 by the company Evonik-Degussa.

According to the present invention, particularly preferred silica particles other than the hydrophobic silica aerogel particle include porous silica microspheres, especially those sold under the names Sunsphere® H53 and Sunsphere® H33 by the company Asahi Glass.

The at least two fillers for use in the composition of the present invention may be present in the composition in a total amount ranging from about 0.1 to about 10% by weight, preferably ranging from about 0.5 to about 5% by weight and more preferably ranging from about 0.5 to about 4% by weight, based on the total weight of the composition, including all ranges and subranges therebetween.

For example, the at least two fillers for use in the composition of the present invention may be present in a total amount of 1% by weight, or at 1.5% by weight, or at 2.0% by weight, or at 2.5% by weight, or at 3% weight, or at 4% weight, or at 5% weight, all weights being based on the total weight of the composition.

In certain embodiments, the at least two fillers in the compositions of the present invention comprise two different platelet type fillers. These platelet type fillers may be chosen from boron nitride, barium sulfate, bismuth oxychloride, alumina and composite powders based on titanium oxide and substrate like talc, mica, barium sulfate, boron nitride, bismuth oxychloride, alumina, and mixtures thereof.

In preferred embodiments, the two platelet type fillers comprise boron nitride and bismuth oxychloride.

In other embodiments, the compositions of the present invention employ two different fillers, that is, a platelet type filler and a silica particle other than the hydrophobic silica aerogel particle. The platelet type fillers may be chosen from boron nitride, barium sulfate, bismuth oxychloride, alumina and composite powders based on titanium oxide and substrate like talc, mica, barium sulfate, boron nitride, bismuth oxychloride, alumina, and mixtures thereof. Preferably, the platelet type fillers are chosen from boron nitride and bismuth oxychloride.

In some embodiments, when the two fillers of the compositions of the present invention comprise a platelet type filler and a silica particle other than the hydrophobic silica aerogel particle, the platelet type filler comprises boron nitride. In other embodiments, when the two fillers of the compositions of the present invention comprise a platelet type filler and a silica particle other than the hydrophobic silica aerogel particle, the platelet type filler comprises bismuth oxychloride.

In yet other embodiments, the compositions of the present invention employ three different fillers, that is, two platelet type fillers comprising boron nitride and bismuth oxychloride and a silica particle other than the hydrophobic silica aerogel particle.

In some embodiments, the platelet type fillers are present in an amount of from about 0.5 to about 3% by weight, or from about 1 to about 2% by weight, based on the total weight of the composition.

In other embodiments, the silica particle other than the hydrophobic silica aerogel particle is present in an amount of from about 0.5 to about 3% by weight, or from about 1 to about 2% by weight, based on the total weight of the composition.

According to preferred embodiments, the at least one silicone material comprising concave and/or annular particles, the at least one hydrophobic silica aerogel particle, and the at least two fillers selected from the group comprising platelet type fillers and silica particles other than the hydrophobic silica aerogel particles are present in a total amount ranging from greater than 4% to about 10% by weight, preferably, from about 4.5% to about 8% by weight, more preferably, from about 5% to about 8% by weight, and even more preferably, from about 6% to about 8% by weight, based on the total weight of the composition of the present invention, including all ranges and subranges therebetween.

In other preferred embodiments of the present invention, the at least one silicone material comprising concave and/or annular particles is present in an amount ranging from about 1% to about 2% by weight, the at least one hydrophobic silica aerogel particle is present in an amount ranging from about 1% to about 2% by weight, and the at least two fillers selected from the group comprising platelet type fillers and silica particles other than the hydrophobic silica aerogel particles are present in a total amount ranging from about 2% to about 4% by weight, all weights being based on the total weight of the composition of the present invention, including all ranges and subranges therebetween.

In yet other preferred embodiments of the present invention, the at least one silicone material comprising concave and/or annular particles, the at least one hydrophobic silica aerogel particle, and the at least two fillers selected from the group comprising platelet type fillers and silica particles other than the hydrophobic silica aerogel particles are employed in a total amount of greater than 4% by weight, based on the total weight of the composition of the present invention.

### Fatty Substances

The compositions of the present invention comprise at least one fatty substance chosen from fatty acids and fatty alcohols.

Preferably, the at least one fatty substance chosen from fatty acids and fatty alcohols is a liquid at room temperature (25 degrees C.) and at atmospheric pressure (760 mmHg).

The fatty alcohols may be chosen from linear, branched or cyclic, saturated or unsaturated, fatty alcohols comprising 6 to 50 carbon atoms and comprising one or more OH, optionally comprising one or more NH2.

Mention may in particular be made of: —linear or branched, saturated or unsaturated, C6-050, especially C6-C32, in particular C8-C28, monoalcohols, and in particular isostearyl alcohol, cetyl alcohol, cetearyl alcohol, oleyl alcohol, isopalmitoyl alcohol, lauryl alcohol, myristyl alcohol, 2-butyloctanol, 2-hexyldecanol, 2-octyldecanol, 2-octyldodecanol, 2-octyltetradecanol, 2-decyl-tetradecanol, 2-dodecylhexadecanol, and in particular the alcohols sold under the name Jarcol™ by the company Jarchem Industries, such as Jarcol™ 1-12, Jarcol™ 1 -16, Jarcol™ I-20 and Jarcol™ I-24;
linear or branched, saturated or unsaturated, C6-050, especially C6-C4o, in particular C8-C38, and especially branched C32-C36, diols, and in particular the commercial product Pripol™ 2033 from Uniqema;
linear or branched, saturated or unsaturated, C6-050, especially C6-C32, in particular C8-C28, triols, and in particular phytantriol;

Preferred fatty alcohols of the present invention may be chosen from cetyl alcohol, cetearyl alcohol, isostearyl alcohol, and oleyl alcohol.

The fatty acids of the present invention may be chosen from the acids of formula RCOOH, in which R is a saturated or unsaturated, linear or branched radical preferably comprising from 7 to 39 carbon atoms.

Preferably, R is a C7-C29 alkyl or C7-C29 alkenyl group and better still a C12-C24 alkyl or C12-C24 alkenyl group. R may be substituted with one or more hydroxyl groups and/or one or more carboxyl groups.

The fatty acids may be chosen from C12-C22 higher fatty acids, such as stearic acid, myristic acid, oleic acid, linoleic acid, linolenic acid, lauric acid, palmitic acid, and mixtures thereof.

In preferred embodiments, the fatty substances of the compositions of the present invention comprise fatty acids.

In other preferred embodiments, the fatty substances of the compositions of the present invention comprise fatty acids and fatty alcohols.

The fatty sub stance(s) are present in the composition in an amount ranging from about 0.1 to about 20% by weight, preferably in an amount ranging from about 0.25 to about 10% by weight, more preferably still in an amount ranging from about 0.25 to about 5% by weight, and even more preferably still in an amount ranging from about 0.5 to about 3% by weight, based on the total weight of the composition, including all ranges and subranges therebetween.

### Emulsifying Agents

The compositions according to the invention comprise at least one emulsifying agent is chosen from oxyethylenated and/or oxypropylenated ethers of glycerol, oxyethylenated and/or oxypropylenated ethers of fatty alcohols, fatty acid esters of polyethylene glycol, fatty acid esters of oxyethylenated and/or oxypropylenated glyceryl ethers, fatty acid esters of polyols and mixtures thereof.

According to the invention, the at least one emulsifying agent is appropriately chosen to obtain a composition that is an emulsion, preferably, an oil-in-water emulsion. In particular, the emulsifying agent has, at 25 degrees centigrade, an HLB (hydrophilic-lipophilic balance), in the Griffin sense, of greater than or equal to 8.

The emulsifying agents of the present invention comprise nonionic surfactants and are further described as follow:

a) nonionic surfactants with an HLB of greater than or equal to 8 at 25 degrees centigrade, used alone or as a mixture; mention may be made especially of:
oxyethylenated and/or oxypropylenated ethers (which may comprise from 1 to 150 oxyethylene and/or oxypropylene groups) of glycerol;
oxyethylenated and/or oxypropylenated ethers (which may comprise from 1 to 150 oxyethylene and/or oxypropylene groups) of fatty alcohols (especially of C8-C24 and preferably C12-Ci8 alcohol), such as oxyethylenated cetearyl alcohol ether containing 30 oxyethylene groups (CTFA name Ceteareth-30), oxyethylenated stearyl alcohol ether containing 20 oxyethylene groups (CTFA name Steareth-20) and the oxyethylenated ether of the mixture of C12-C15 fatty alcohols comprising 7 oxyethylene groups (CTFA name C12-15 Pareth-7) sold under the name Neodol 25-7(R) by Shell Chemicals;
fatty acid esters (especially of a C8-C24 and preferably Ci6-C22 acid) of polyethylene glycol (which may comprise from 1 to 150 ethylene glycol units), such as PEG-100 stearate or PEG-50 stearate and PEG-40 monostearate sold under the name Myrj™ 52P by the company ICI Uniqema, or else PEG-30 glyceryl stearate sold in particular under the name Tagat® S by the company Evonik Goldschmidt, or else PEG-150 distearate sold in particular under the name KESSCO® PEG 6000 DS by the company ITALMATCH CHEMICALS ARESE;
fatty acid esters (especially of a C8-C24 and preferably Ci6-C22 acid) of oxyethylenated and/or oxypropylenated glyceryl ethers (which may comprise from 1 to 150 oxyethylene and/or oxypropylene groups), for instance PEG-200 glyceryl monostearate sold under the name Simulsol® 220 ™ by the company SEPPIC; glyceryl stearate polyethoxylated with 30 ethylene oxide groups, for instance the product Tagat® S sold by the company Evonik Goldschmidt, glyceryl oleate polyethoxylated with 30 ethylene oxide groups, for instance the product Tagat® 0 sold by the company Evonik Goldschmidt, glyceryl cocoate polyethoxylated with 30 ethylene oxide groups, for instance the product Varionic® LI 13 sold by the company Sherex, glyceryl isostearate polyethoxylated with 30 ethylene oxide groups, for instance the product Tagat® L sold by the company Evonik Goldschmidt, and glyceryl laurate polyethoxylated with 30 ethylene oxide groups, for instance the product Tagat® I from the company Evonik Goldschmidt;

b) nonionic surfactants with an HLB of less than 8 at degrees centigrade, optionally combined with one or more nonionic surfactants with an HLB of greater than 8 at 25 degrees centigrade, such as those mentioned above; mention may be made especially of:
oxyethylenated and or oxypropylenated ethers (which may comprise from 1 to 150 oxyethylene and/or oxypropylene groups) of fatty alcohols (especially of C8-C24 and preferably C12-C)g alcohols) such as the oxyethylenated ether of stearyl alcohol containing two oxyethylene groups (CTFA name Steareth-2);
fatty acid esters (especially of a C8-C24 and preferably Ci6-C22 acid) of polyols, especially of glycerol or of sorbitol, such as glyceryl stearate, glyceryl stearate such as the product sold under the name Tegin® M by the company Evonik Goldschmidt, glyceryl laurate such as the product sold under the name Imwitor® 312 by the company Hiils, polyglyceryl-2 stearate, sorbitan tristearate or glyceryl ricinoleate.

According to one particular embodiment of the invention, the at least one emulsifying agent of the present invention comprises glyceryl esters and polyethylene glycol esters of fatty acids.

According to another particular embodiment of the invention, the at least one emulsifying agent of the present invention is chosen from PEG-150 distearate sold under the name of Kessco PEG® 6000 DS from ITALMATCH CHEMICALS ARESE, Glyceryl stearate (and) PEG-100 Stearate (for example such as the product sold under name ARLACEL™ 165 from CRODA); and mixtures thereof.

The composition according to the invention may contain from about 0.1 to about 10% by weight, or from about 0.25 to about 5% by weight of total emulsifying agent(s), based on the total weight of the said composition, including all ranges and subranges therebetween.

### Thickening Agents

The at least one thickening agent of the present invention are preferably chosen from polymers of natural origin and may include, for example, thickening polymers comprising at least one sugar unit, for instance nonionic guar gums, optionally modified with C1-C6 hydroxyalkyl groups; biopolysaccharide gums of microbial origin, such as scleroglucan gum or xanthan gum; gums derived from plant exudates, such as gum arabic, ghatti gum, karaya gum, gum tragacanth, carrageenan gum, agar gum and carob gum; pectins; alginates; starches; hydroxy(C1-C6)alkylcelluloses and carboxy(C1-C6)alkylcelluloses.

It should be noted that the term “sugar unit” denotes a monosaccharide (i.e. monosaccharide or oside or simple sugar) portion, an oligosaccharide portion (short chains formed from a sequence of monosaccharide units, which may be different) or a polysaccharide portion [long chains consisting of monosaccharide units, which may be different, i.e. polyholosides or polyosides]. The saccharide units may also be substituted with alkyl, hydroxyalkyl, alkoxy, acyloxy or carboxyl radicals, the alkyl radicals containing from 1 to 4 carbon atoms.

Non-limiting examples of nonionic, unmodified guar gums that may be used in various embodiments include Guargel D/15 (Noveon); Vidogum GH 175 (Unipectine), Meypro-Guar 50 and Jaguar C (Meyhall/Rhodia Chimie). Non-limiting examples of modified nonionic guar gums include Jaguar HP8, HP60, HP120, DC 293 and HP 105 (Meyhall/Rhodia Chimie); Galactasol 4H4FD2 (Aqualon).

Among these gums, mention will be made of scleroglucans such as, for example, Actigum CS from Sanofi Bio Industries; Amigel from Alban Muller International, and also the glyoxal-treated scleroglucans described in FR2633940); xanthan gums, for instance Keltrol®, Keltrol® T, Keltrol® Tf, Keltrol® Bt, Keltrol® Rd, Keltrol® Cg (Nutrasweet Kelco), Rhodicare® S and Rhodicare® H (Rhodia Chimie); starch derivatives, for instance Primogel® (Avebe); hydroxyethylcelluloses such as Cellosize® QP3L, QP4400H, QP30000H, HEC30000A and Polymer PCG10 (Amerchol), Natrosol® 250HHR, 250MR, 250M, 250HHXR, 250HHX, 250HR, HX (Hercules) and Tylose® H1000 (Hoechst); hydroxypropylcelluloses, for instance Klucel® EF, H, LHF, MF and G (Aqualon); carboxymethylcelluloses, for instance Blanose® 7M8/SF, refined 7M, 7LF, 7MF, 9M31F, 12M31XP, 12M31P, 9M31XF, 7H, 7M31, 7H3SXF (Aqualon), Aquasorb® A500 (Hercules), Ambergum® 1221 (Hercules), Cellogen® HP810A, HP6HS9 (Montello) and Primellose® (Avebe).

In other preferred embodiments, the at least one thickening agent are chosen from nonionic polymers which include, but are not limited to, nonionic cellulose derivatives such as hydroxyethylcelluloses modified by groups comprising at least one hydrophobic chain, such as alkyl, arylalkyl or alkylaryl groups, or their blends, and in which the alkyl groups are, for example, C8-C22 alkyl groups, such as the product Natrosol® Plus Grade 330 CS (C16 alkyls) sold by Aqualon or the product Bermocoll® EHM 100 sold by Berol Nobel.

Cellulose derivatives modified by alkylphenyl polyalkylene glycol ether groups may also be chosen, such as the product Amercell® Polymer HM-1500 sold by Amerchol.

In certain exemplary embodiments, the at least one thickening agent is chosen from cellulose derivatives, polysaccharides, gums, clays, fumed silica, acrylates, polyacrylamides, crosslinked polyacrylic acids, crosslinked acrylamide polymers and copolymers, crosslinked methacryloyloxyethltrimethyl-ammonium chloride homopolymers, and associative polymers. Said rheology-modifying agents may include, in particular embodiments, xanthan gum, gum arabic, ghatti gum, karaya gum, gum tragacanth, carrageenan gum, agar gum, carob gum, pectins, alginates, starches, hydroxy(C1-C6)alkylcelluloses, carboxy(C1-C6)alkylcelluloses, and mixtures thereof.

In preferred embodiments, the at least one thickening agent is nonionic or is an uncharged compound. Without intending to be bound by theory, it is believed that a thickening agent comprising a charged compound could adversely interact with the dyes compounds, thereby affecting the solubility and stability of the dye compounds in the compositions of the present disclosure.

In particularly preferred embodiments, the at least one thickening agent is chosen from xanthan gum, carrageenan gum, guar gum, and mixtures thereof.

The at least one thickening agent of the present disclosure may be employed in the composition of the present invention in an amount ranging from, for example, about 0.05 to about 10% by weight, preferably from about 0.1 to about 8% by weight and more preferably from about 0.25 to about 5% by weight, based on the total weight of the composition, including all ranges and subranges therebetween.

### Cosmetically Acceptable Medium

Besides the compounds indicated previously, a composition according to the invention comprises a cosmetically acceptable medium. In preferred embodiments, the cosmetically acceptable medium comprises water

The term “cosmetically acceptable medium” is intended to denote a medium that is particularly suitable for applying a composition according to the invention to the skin.

The cosmetically acceptable medium is generally adapted to the nature of the support onto which the composition is to be applied, and also to the form in which the composition is to be packaged.

A composition of the invention may be a dispersion or an emulsion.

A dispersion may be made as an aqueous phase or as an oily phase.

An emulsion may have an oily or aqueous continuous phase. Such an emulsion may be, for example, an inverse (W/O) emulsion or a direct (O/W) emulsion, or alternatively a multiple emulsion (W/O/W or O/W/O).

In the case of emulsions, direct (O/W) emulsions are preferred.

### Aqueous Phase

The composition according to the invention may comprise an aqueous phase.

The aqueous phase comprises water. A water that is suitable for use in the invention may be a floral water such as cornflower water and/or a mineral water such as Vittel water, Lucas water or La Roche Posay water and/or a spring water.

The aqueous phase may also comprise water-miscible organic solvents (at room temperature: 25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C1-C4)alkyl ethers, mono-, di- or triethylene glycol (C1-C4)alkyl ethers, and mixtures thereof.

The aqueous phase may also comprise stabilizers, for example sodium chloride, magnesium dichloride or magnesium sulfate.

The aqueous phase may also comprise any water-soluble or water-dispersible compound that is compatible with an aqueous phase, such as gelling agents, film-forming polymers, thickeners or surfactants, and mixtures thereof.

In particular, a composition of the invention may comprise an aqueous phase in a content ranging from 1% to 80% by weight, especially from 5% to 50% and more particularly from 10% to 45% by weight, based on the total weight of the composition, including all ranges and subranges therebetween.

### Fatty Phase

A cosmetic composition in accordance with the present invention may comprise at least one liquid and/or solid fatty phase.

According to one embodiment, the composition according to the present invention is in the form of an emulsion.

In particular, a composition of the invention may comprise at least one liquid fatty phase, especially at least one oil as mentioned below.

The term “oil” means any fatty substance that is in liquid form at room temperature (20-25° C.) and at atmospheric pressure.

A composition of the invention may comprise a liquid fatty phase in a content ranging from about 1% to about 90%, in particular from about 5% to about 80%, in particular from about 10% to about 70% and more particularly from about 20% to about 50% by weight relative to the total weight of the composition.

The oily phase that is suitable for preparing the cosmetic compositions according to the invention may comprise hydrocarbon-based oils, silicone oils, fluoro oils or non-fluoro oils, or mixtures thereof.

The oils may be volatile or non-volatile.

They may be of animal, plant, mineral or synthetic origin.

The term “non-volatile oil” means an oil that remains on the skin at room temperature and atmospheric pressure. More specifically, a non-volatile oil has an evaporation rate strictly less than 0.01 mg/cm2/min.

To measure this evaporation rate, 15 g of oil or of oil mixture to be tested are placed in a crystallizing dish 7 cm in diameter, which is placed on a balance in a large chamber of about 0.3 m3 that is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit of area (cm2) and per unit of time (minutes).

The term “volatile oil” means any non-aqueous medium that is capable of evaporating on contact with the skin or the lips in less than one hour, at room temperature and atmospheric pressure. The volatile oil is a cosmetic volatile oil, which is liquid at room temperature. More specifically, a volatile oil has an evaporation rate of between 0.01 and 200 mg/cm2/min, limits included.

For the purposes of the present invention, the term “silicone oil” means an oil comprising at least one silicon atom, and especially at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

### Volatile Oils

The volatile oils may be chosen from hydrocarbon-based oils containing from 8 to 16 carbon atoms, and especially C8-C16 branched alkanes (also known as isoparaffins), for instance isododecane (also known as 2,2,4,4,6-pentamethylheptane), isodecane and isohexadecane, for instance the oils sold under the trade names Isopar® or Permethyl®.

Volatile oils that may also be used include volatile silicones, for instance volatile linear or cyclic silicone oils, especially those with a viscosity of less than or equal to 8 centistokes (cSt) (8×10-6 m2/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

According to one embodiment, a composition of the invention may comprise from about 1% to about 80% by weight, or even from about 5% to about 70% by weight, or even from about 10% to about 60% by weight and especially from about 15% to about 50% by weight of volatile oil relative to the total weight of the composition.

Non-volatile oils

The non-volatile oils may be chosen especially from non-volatile hydrocarbon-based, fluoro and/or silicone oils.

Non-volatile hydrocarbon-based oils that may especially be mentioned include:
hydrocarbon-based oils of animal origin, such as perhydrosqualene,
hydrocarbon-based oils of plant origin, such as phytostearyl esters, such as phytostearyl oleate, phytostearyl isostearate and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, winter squash oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camelina oil, canola oil, carrot oil, safflower oil, flax oil, rapeseed oil, cotton oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant pip oil, kiwi seed oil, grapeseed oil, pistachio oil, winter squash oil, pumpkin oil, musk rose oil, sesame oil, soybean oil, sunflower oil, castor oil and watermelon seed oil, and mixtures thereof, or alternatively caprylic/capric acid triglycerides, such as those sold by the company Stearineries Dubois or those sold under the names Miglyol® 810, 812 and 818 by the company Dynamit Nobel,
linear or branched hydrocarbons of mineral or synthetic origin, such as liquid paraffins and derivatives thereof, petroleum jelly, polydecenes, polybutenes, hydrogenated polyisobutene such as Parleam, and squalane;
synthetic ethers containing from 10 to 40 carbon atoms;
oils of high molar mass, in particular having a molar mass ranging from about 400 to about 10 000 g/mol, in particular from about 650 to about 10 000 g/mol, in particular from about 750 to about 7500 g/mol and more particularly ranging from about 1000 to about 5000 g/mol. As oils of high molar mass that may be used in the present invention, mention may especially be made of oils chosen from:
lipophilic polymers,
silicone oils,
oils of plant origin, and
mixtures thereof;
optionally partially hydrocarbon-based and/or silicone fluoro oils, for instance fluorosilicone oils, fluoropolyethers and fluorosilicones as described in document EP-A-847 752;
silicone oils, for instance linear or cyclic non-volatile polydimethylsiloxanes (PDMS); polydimethylsiloxanes comprising alkyl, alkoxy or phenyl groups, which are pendant or at the end of a silicone chain, these groups containing from 2 to 24 carbon atoms; phenyl silicones, for instance phenyl trimethicones, phenyl dimethicones, phenyl trimethylsiloxy diphenyl siloxanes, diphenyl dimethicones, diphenyl methyldiphenyl trisiloxanes and 2-phenylethyl trimethylsiloxy silicates, and
mixtures thereof.

According to one particular embodiment, the fatty phase of the composition according to the invention can contain only volatile compounds.

According to another particular embodiment, the fatty phase of the composition according to the invention can contain non-volatile oils, preferably, in a total amount of not more than about 20% by weight, preferably, not more than about 10% by weight, or more preferably, not more than about 5% by weight, based on the total weight of the composition.

According to certain embodiments, the fatty substance of the present invention chosen from fatty alcohols and fatty acids are present in the fatty phase of the composition.

### Colorants

A composition according to the invention may also comprise at least one colorant.

The amount of colorant(s) in the composition of the invention will generally range from 0 to about 5% by weight of total weight of the composition, in particular from 0.5 to 3% by weight of total weight of the composition.

In a particular embodiment, the composition will contain a low amount of titanium dioxide, ie less than 5% by weight, preferably less than 3% by weight of titanium dioxide.

A composition in accordance with the invention may incorporate at least one colorant chosen from mineral or organic pigments conventionally used in cosmetic compositions, liposoluble or water-soluble dyes, and mixtures thereof.

The term “pigments” should be understood to mean white or coloured, inorganic or organic particles which are insoluble in an aqueous solution and are intended for colouring and/or opacifying the resulting film.

As inorganic pigments that can be used in the invention, mention may be made of titanium oxides, zirconium oxides or cerium oxides, and also zinc oxides, iron oxides or chromium oxides, ferric blue, manganese violet, ultramarine blue and chromium hydrate. According to one particular mode of the invention, the mineral pigments will be chosen from iron oxides and titanium oxides, and mixtures thereof.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The colorant may also comprise a pigment having a structure which may be, for example, of the type such as silica microspheres containing iron oxide. An example of a pigment having this structure is the product sold by the company Miyoshi under the reference PC Ball PC-LL-100 P, this pigment being constituted of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP 0 542 669, EP 0 787 730, EP 0 787 731 and WO 96/08537.

The cosmetic composition according to the invention may also comprise water-soluble or fat-soluble dyes. The liposoluble dyes are, for example, Sudan red, DC Red 17, DC Green 6, β-carotene, soybean oil, Sudan brown, DC Yellow 11, DC Violet 2, DC Orange 5 and quinoline yellow. The water-soluble dyes are, for example, beetroot juice and caramel.

### Additional Fillers

A composition in accordance with the invention may also comprise at least one additional filler, of organic or mineral nature, making it possible especially to give it additional matt-effect or homogenizing/covering properties, and/or improved stability with regard to exudation and migration-resistance properties after application.

The fillers used in the compositions according to the present invention may be in lamellar, globular or spherical form, in the form of fibres or in any other intermediate form between these defined forms.

Examples of mineral fillers that may be mentioned include kaolin, calcium carbonate, magnesium carbonate, hydroxyapatite, glass or ceramic microcapsules.

Examples of organic fillers that may be mentioned include polyethylene powder or polymethyl methacrylate powder, polytetrafluoroethylene (Teflon) powders, lauroyllysine, hexamethylene diisocyanate/trimethylol hexyl lactone copolymer powder (Plastic Powder® from Toshiki), silicone resin microbeads (for example Tospearl® from Toshiba), natural or synthetic micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, and polyurethane powders, in particular crosslinked polyurethane powders comprising a copolymer, the said copolymer comprising trimethylol hexyl lactone. It may in particular be a hexamethylene diisocyanate/trimethylol hexyl lactone polymer. Such particles are especially commercially available, for example, under the name Plastic Powder D-400® or Plastic Powder D-800® from the company Toshiki, and mixtures thereof.

### Additives

A composition according to the invention may also comprise any additive usually used in the field under consideration, chosen, for example, from waxes, dispersants, antioxidants, essential oils, preserving agents, fragrances, neutralizers, antiseptics, UV-screening agents, cosmetic active agents, such as vitamins, moisturizers, emollients or collagen-protecting agents, and mixtures thereof.

It is a matter of routine operations for a person skilled in the art to adjust the nature and amount of the additives present in the compositions in accordance with the invention such that the desired cosmetic properties and stability properties thereof are not thereby affected.

A cosmetic composition of the invention may be in the form of a skin makeup product, in particular a foundation, a hot-cast foundation product, a body makeup product, a concealer, or an eyeshadow. It may be in the form of cream, lotion or soft paste.

A care composition according to the invention may in particular be a sunscreen or a moisturizing composition.

Preferably, the composition according to the invention is in the form of a fluid primer or base or a fluid foundation. When the composition is a foundation, it further contains at least one colorant as described above in an amount sufficient to provide additional color to or change the color of the skin.

In a particular embodiment, the composition is an emulsion. Such emulsions will generally be comprised of the liquid fatty phase (also known as an oil phase) and the aqueous phase as described earlier.

The invention also concerns a process comprising a step of applying at least one layer of the composition according to the invention, onto the skin, in particular the skin of the face.

In a particular embodiment, the composition is applied alone or as a base or primer under a skin care product or a makeup product.

The process is particularly intended to minimize or decrease the visibility of skin imperfections, in particular, the pores, uneven skin tone or color and skin discoloration.

In accordance with these preceding preferred embodiments, the compositions of the present invention are applied topically to the desired area in an amount sufficient to minimize the visibility of skin imperfections, care for and/or enhance the appearance of skin. The compositions may be applied to the desired area as needed, preferably once or twice daily, more preferably once daily. When the composition is applied onto skin as a primer or a base, then it may be allowed to dry before a subsequent composition is applied onto the skin. Preferably, the composition is allowed to dry for about 1 minute or less, more preferably for about 45 seconds or less. The composition is preferably applied to the desired area that is dry or has been dried prior to application.

The compositions of the present invention, such as for example, those which are employed as a foundation containing colorants, may also be applied onto skin to which a basecoat or primer has been previously applied.

The composition according to the present disclosure may be manufactured by the known processes generally used in cosmetics and personal care products.

The composition may be packaged in a jar or a bottle, which may be equipped with a pump mechanism for delivering the composition onto the skin or the fingers/hand.

The packaged composition may also be accompanied by an applicator device which allows the composition to be taken up and also allows the composition taken up to be deposited on the skin.

When a composition according to the present invention is applied onto skin, the composition has a homogenizing effect on skin, that is, the composition eves the skin tone or color and/or produces a blurring/haze or soft focus effect on the skin such that the appearance of discolorations, blemishes, pores, fine lines or wrinkles is minimized.

At the same time, the composition of the present invention does not produce an undesirable whitish sheen or film on the skin but is transparent/translucent such that the skin has a natural appearance.

A homogenizing effect according to the present invention can also be described in terms of the homogenizing power of the composition as described below.

Protocol for Measuring or Evaluating the Efficacy of the Compositions According to the Invention with Respect to Minimizing the Visibility of Skin Imperfections.

The compositions of the invention are in particular characterized by a parameter called homogenizing power and a second parameter, transparency, Tr, according to the protocol described hereinafter.

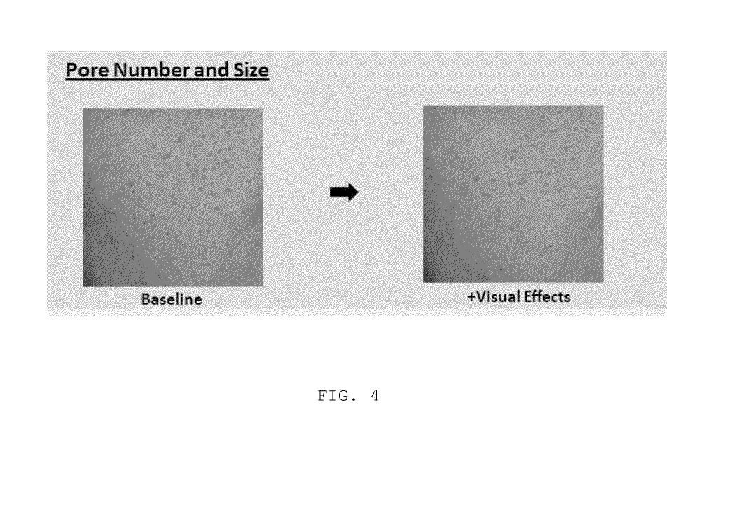

The color measurements (chroma C, luminance L and hue h), before and after application of a composition according to the invention, are carried out on a contrast card representative of the skin of which the lightness is between 30 and 40. In particular, these measurements are carried out on a contrast card 32 (e.g., (Prufkarte type 24/5—250 cm2, sold by the company Erichsen) as defined in application US2005/0025728 (EP1433461) and represented in FIG. 4 of said application (reproduced in FIG. 1 of the present application).

Represented on this FIG. 1 are three contrast cards, 30, 31 and 32, brought together on the same support 33.

The support 33 comprises a white border 34 which extends all around the contrast cards 30, 31 and 32 which are placed side by side.

The contrast card 32 comprises a square colored area B11+ and four rectangular colored areas B11, B12, B12+ and XXX.

The various areas B11, B11+, B12, B12+ and XXX are preferably made by printing with inks chosen such that these areas appear to an observer to be of the same color under at least two differing illuminants, as described in European patent application EP1212961.

The support 33 is, for example, a matt sheet of paper of 130 g/cm2, for inkjet printing.

The contrast card 32 may be representative of skin of which the lightness is between 30 and 40.

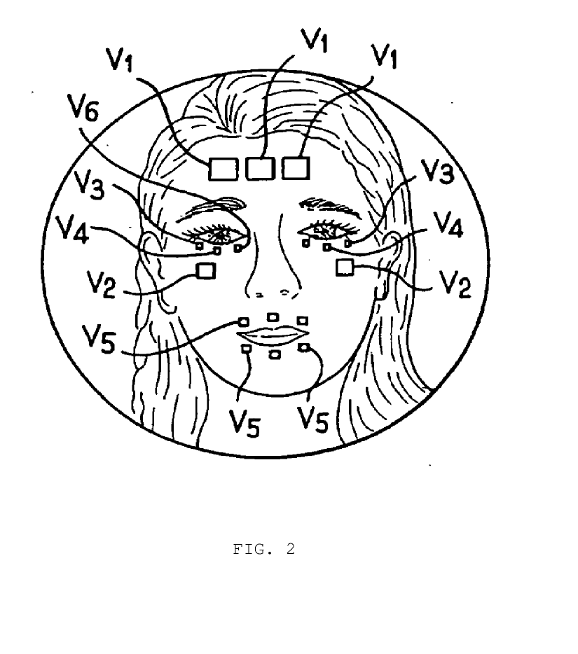

Thus, the various areas B11+, B11, B12, B12+ and XXX can correspond to average colors observed on a panel of individuals having dark skin with a dark complexion as regards various regions of the face, represented in FIG. 2 of the present application.

The region B11+ can correspond to the average of the color measured on the forehead in the areas V1 averaged with that measured on the cheekbones, in the areas V2.

The area B11 can correspond to the color measured on the cheekbones, in the area V2, the area B12 to that measured on the forehead, in the areas V1, the area B12+ to the average color between that measured on the outer dark shadows in the areas V3, that measured in the dark shadows in the middle in the areas V4 and that measured around the lips in the areas V5. The area XXX can correspond to the color of the inner dark shadows, measured in the areas V6.

The contrast card 32 is characterized, for each area, by the following values:

| Area | B11+ | XXX | B12+ | B12 | B11 |
|---|---|---|---|---|---|
| L* | 36.7 | 29.63 | 32.98 | 35.66 | 38.43 |
| C* | 19.81 | 15.06 | 17.29 | 19.78 | 21.76 |
| h | 47.34° | 40.34° | 44.64° | 46.32° | 46.51° |

Where L* is the lightness, and C* the saturation, and h the hue, in the CIE 1976 colorimetric space.

The measurements were carried out with a MINOLTA® spectrocolorimeter, of reference CM2002, in reflection mode, specular included, medium aperture.

The spectral reflectances measured are those given in Table I of application EP 1433461, these values being given by way of non-limiting example only.

The values of L* and h are given to within 15 percent, or even to within 12.5 percent, better still to within 10 percent, or even to within 7.5 percent, or even to within 5 percent, and the values of C* are given to within 25 percent, better still to within 20 percent, even better still to within percent, even to within 10 percent or even to within 5 percent.

In particular, the values of L*, h and C* are given to within 15 percent. These color measurements are carried out using a KONICA MINOLTA CR-400 chromameter. It is a tristimulus colorimeter which contains filters, detectors and a filtered lamp. The measurements are carried out in the colorimetric space: L, C, h. The operating conditions are the following:
Standard observer CI E 1964 10 degrees
Illuminant CIE type D65
Measurement geometry d/0, variant of d/8. The instrument is equipped with an optical device which produces diffuse light, placed in a spherical cavity coated with a white layer which induces a multiple reflection of the light. An anti-glare trap eliminates the effects of glare of the surface of the sample.

Initially, a “white plate” calibration of the instrument is carried out.

The average color shift is obtained from the measurement of the colorimetric parameters L, C and h on the area B11+ of the bare (untreated) contrast card 32. The average color shift from the untreated card is the average difference between the area B11+, B12 and B11+, and so forth and is calculated as:

$$Δ {E}_{0} m = \frac{Σ [ Δ Eo - n ]}{n}$$

$$with Δ {E}_{0 - n} = \sqrt{{( L {1}^{*} - L {n}^{*} )}^{2} + {( a {1}^{*} - {an}^{*} )}^{2} + {( b {1}^{*} - {bn}^{*} )}^{2}}$$

$$wherein \text{:}$$

$$n = 1 , 2 , … , n$$

L*—Lightness (34-41), ΔL—Light Variation typically has values of 0.5 to 4. ΔL* yield values that will not make the person have lighter skin but not look grayish or dull.

a*—red/green axis, values between 9 and 15.

b*—blue/yellow axis, values are typically between 28-42 values.

In parallel, the composition to be evaluated is spread on a transparent film by means of an applicator (e.g. automatic applicator from Braive instruments) (wet thickness 50 microns). The spreadings are then placed in a thermostated and ventilated oven for 24 hours at 37 degrees centigrade.

Once dried, these films are placed for evaluation on the area B11+ of the contrast card 32. The parameters ΔC and ΔL are defined as the respective variations in the chroma C and in the luminance L of the area B11+ after application of a cosmetic composition according to the invention.

Once spread into a thin film and dried, the compositions of the invention can be characterized by the following optical properties:

−25<ΔC<−2 where C is the chroma

5<ΔL<30 where L is the luminance

50<percent Tr<70, in particular 50<percent Tr<68, or even 50<percent Tr<66 where percent Tr is the transparency.

The color shift on the treated contrast card is measured to obtain ΔEₜᵣₑₐₜₘₑₙₜ.

Homogenizing power, HP, is calculated as:

HP=ΔE₀m/ΔEₜᵣₑₐₜₘₑₙₜ

### Protocol for Measuring the Opacity of the Compositions According to the Invention.

The transparency, Tr, of the compositions is measured according to the following protocol: Dried films prepared according to the above-described method are placed for evaluation on a contrast card. The opacity is then measured using the Minolta CR-400 chromameter from the values Y of the black and white parts obtained in the tristimulus system (X, Y, Z).

The transparency value is obtained from the following equations:

[1−(Y black area/Y white area)]×100=% Opacity and

% Transparency=100−% Opacity

If the film is totally opaque, the transparency is equal to zero. Thus, the higher that % transparency, the more transparent the film is.

Data can be further analyzed by plotting on a graph, transparency as a function of homogenizing power.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

All compositions are written with percentages by weight. They were prepared according to the same protocol: mix oil phase ingredients together and heat up to 60-80° C. until wax is melted, disperse pigments and fillers inside the oil phase then proceed to emulsification by adding water phase ingredients.

### Example 1

TABLE 1
Inventive Composition; oil-in-water emulsion

| | | Inventive |
|---|---|---|
| PHASE | INCI US | Composition |
| A | ISOPROPYL LAUROYL SARCOSINATE | 4.00 |
| A | GLYCERYL STEARATE (and) PEG-100 STEARATE | 0.60 |
| A | STEARIC ACID | 1.00 |
| A | CETYL ALCOHOL | 0.50 |
| A | TOCOPHERYL ACETATE | 0.50 |
| A | PENTAERYTHRITYL TETRA-DI-T-BUTYL | 0.10 |
| | HYDROXYHYDROCINNAMATE | |
| B | WATER | 30.00 |
| B | GLYCERIN | 7.00 |
| B | BUTYLENE GLYCOL | 7.00 |
| B | PHENOXYETHANOL | 0.50 |
| B | DISODIUM EDTA | 0.15 |
| C | WATER | 25.00 |
| D | DIMETHICONE | 5.00 |
| D | XANTHAN GUM | 0.25 |
| D | SODIUM HYALURONATE | 0.05 |
| E | POLYACRYLAMIDE (and) C13-14 ISOPARAFFIN | 2.00 |
| | (and) LAURETH-7 | |
| F | WATER | 7.35 |
| G | ALCOHOL DENAT. | 3.00 |
| H | METHYLSILANOL/SILICATE CROSSPOLYMER (NLK- | 2.00 |
| | 506 from TAKEMOTO OIL & FAT) | |
| I | SILICA SILYLATE (VM-2270 AEROGEL ® FINE | 2.00 |
| | PARTICLES from DOW CORNING) | |
| J | BORON NITRIDE (from MIZUSHIMA FERROALLOY) | 1.00 |
| K | BISMUTH OXYCHLORIDE (TIMIRON ® LQUID SILVER | 1.00 |
| | from MERCK or Biron ® Liquid Silver from the | |
| | company, EMD Chemicals, Inc) | |

Method of Manufacturing

Formula A was prepared according to the following procedure:

1. In side kettle (beaker), phase A was mixed with heating to 60-65° C.
2. In main kettle, phase B was mixed with heating to 60-65° C. Mixing was continued until phase was uniform.
3. Phase A was added to phase B at 60-65° C. Homogenizing plus macro-mixing was carried out for 15 minutes. Phase C was added with mixing. Heating was discontinued.
4. Phase D was premixed at room temperature, and was added to phase (A+B+C) at 40-50° C. Homogenizing plus macro-mixing was carried out for 10 minutes until gums were well dissolved and lump free.
5. Phase E was added to phase (A+B+C+D). Homogenizing plus macro-mixing was carried out for 5 minutes.
6. Phase F was premixed at room temperature. Phase F was added to phases (A+B+C+D+E) at 25-30° C. Homogenizing plus macro-mixing was carried out for 5 minutes.
7. Phase G was added. Homogenizing plus macro-mixing was carried out for 5 minutes.
8. Batch was stopped and specifications were checked.
9. Phases H, I, J and K were homogenized into the formula using a speed mixer.

### Example 2

### In-Vitro Studies: Homogenizing Power and Transparency Measurements

The homogenizing power and degree of transparency of compositions prepared with different base formulas and varying levels of the fillers and particles of the invention were measured in accordance with the earlier described protocols for measuring homogenizing power and transparency.

TABLE 2
Base formula of the inventive compositions and base formulas
of comparative compositions, oil-in-water emulsions

| | BASE FORMULA | | |
|---|---|---|---|
| INCI NAME | A (*) | B(**) | C(**) |
| DISODIUM EDTA | 0.15 | 0.05 | 0.10 |
| PENTAERYTHRITYL TETRA-DI-T-BUTYL | 0.10 | | |
| HYDROXYHYDROCINNAMATE | | | |
| METHYLSILANOL MANNURONATE | | 6.00 | |
| PHENOXYETHANOL | 0.50 | | 0.50 |
| CHLORPHENESIN | | | 0.25 |
| HYDROLYZED RICE PROTEIN | | 2.00 | |
| PANTHENOL | | 0.30 | |
| TOCOPHERYL ACETATE | 0.50 | 0.04 | |
| PENTAERYTHRITYL | | 1.86 | 2.00 |
| TETRAETHYLHEXANOATE | | | |
| CETYL ALCOHOL | 0.50 | | |
| ISOPROPYL LAUROYL SARCOSINATE | 4.00 | | |
| STEARIC ACID | 1.00 | | |
| GLYCERYL STEARATE (and) PEG-100 | 0.60 | | |
| STEARATE | | | |
| INULIN LAURYL CARBAMATE | | | 0.50 |
| XANTHAN GUM | 0.25 | 0.15 | 0.20 |
| SODIUM HYALURONATE | 0.05 | | |
| POLYACRYLAMIDE (and) C13-14 | 2.00 | 1.10 | |
| ISOPARAFFIN (and) LAURETH-7 | | | |
| AMMONIUM POLYACRYLOYLDIMETHYL | | 0.40 | 0.60 |
| TAURATE | | | |
| AMMONIUM | | | 0.40 |
| ACRYLOYLDIMETHYLTAURATE/ | | | |
| STEARETH-8 METHACRYLATE COPOLYMER | | | |
| DIMETHICONE | | 1.12 | |
| DIMETHICONE | | 1.30 | |
| CYCLOHEXASILOXANE | | 2.42 | |
| DIMETHICONE | 5.00 | | 4.00 |
| DIMETHICONE/VINYL DIMETHICONE | | 2.60 | |
| CROSSPOLYMER (and) DIMETHICONE | | | |
| DIMETHICONE (and) DIMETHICONOL | | 0.49 | 3.00 |
| POLYMETHYLSILSESQUIOXANE | | 0.19 | |
| POLYSILICONE-11 | | | 20.00 |
| ACQUEOUS PHASE INGREDIENTS | | | |
| BUTYLENE GLYCOL | 7.00 | | |
| ALCOHOL DENAT. | 3.00 | 7.50 | 5.00 |
| WATER | 68.35 | 67.50 | 58.45 |
| GLYCERIN | 7.00 | 5.00 | 5.00 |

(*) For inventive compositions
(**)For comparative compositions

The base formula A of the inventive compositions and the comparative base formulas B and C above were used to formulate test compositions containing one or more of the fillers and particles of the invention at various combinations and levels. Base formulas B and C are emulsion compositions which do not contain fatty substances selected from fatty alcohols and fatty acids and the emulsifying agent employed by Base formula A (glyceryl stearate and PEG-100 stearate).

The fillers and particles employed in the test compositions were:

| INCI NAME | COMMERCIAL REFERENCE |
|---|---|
| METHYLSILANOL/SILICATE | NLK-506 from TAKEMOTO OIL & FAT or |
| CROSSPOLYMER | DIAKALYTE ® |
| SILICA SILYLATE | AEROGEL OR DOW CORNING VM-2270 |
| | AEROGEL ® FINE PARTICLES from DOW |
| | CORNING |
| BORON NITRIDE | BORON NITRIDE SHP 3 from MIZUSHIMA |
| | FERROALLOY |
| BISMUTH OXYCHLORIDE | TIMIRON ® LQUID SILVER from MERCK OR |
| | Biron ® Liquid Silver from the |
| | company, EMD Chemicals, Inc |
| SILICA | SOLESPHERE H 51 from ASAHI GLASS |

Note that the raw materials for the fillers and particles are 100%, except for Aerogel, which is 98% active for silica silylate.

TABLE 3
Homogenizing Power and Transparency Values for Compositions Prepared from Base Formula A

| | | | Boron | Bismuth | | Homogenizing | | |
|---|---|---|---|---|---|---|---|---|
| Formula # | NLK-506 | Aerogel | Nitride | Oxychloride | Silica | Power | Stdev | Transparency |
| 1 | 0 | 0 | 0 | 0 | 0 | 0.81 | 0.21 | 88.52% |
| Base | | | | | | | | |
| Formula A | | | | | | | | |

Test Compositions in Base Formula A

| | | | | | | | | |
|---|---|---|---|---|---|---|---|---|
| 2 | 0 | 0 | 0 | 0 | 2 | 0.99 | 0.11 | 88.73% |
| 3 | 0 | 0 | 0 | 1 | 0 | 1.26 | 0.11 | 81.21% |
| 4 | 0 | 0 | 0 | 1 | 2 | 1.87 | 0.18 | 79.52% |
| 5 | 0 | 0 | 1 | 0 | 0 | 1.06 | 0.06 | 86.14% |
| 6 | 0 | 0 | 1 | 0 | 2 | 1.22 | 0.29 | 84.60% |
| 7 | 0 | 0 | 1 | 1 | 0 | 1.23 | 0.23 | 81.87% |
| 8 | 0 | 0 | 1 | 1 | 2 | 1.88 | 0.13 | 73.08% |
| 9 | 0 | 2 | 0 | 0 | 0 | 1.13 | 0.06 | 83.41% |
| 10 | 0 | 2 | 0 | 0 | 2 | 1.24 | 0.06 | 81.58% |
| 11 | 0 | 2 | 0 | 1 | 0 | 1.36 | 0.08 | 85.59% |
| 12 | 0 | 2 | 0 | 1 | 2 | 1.37 | 0.14 | 86.05% |
| 13 | 0 | 2 | 1 | 0 | 0 | 1.65 | 0.15 | 67.97% |
| 14 | 0 | 2 | 1 | 0 | 2 | 1.77 | 0.19 | 81.89% |
| 15 | 0 | 2 | 1 | 1 | 0 | 1.73 | 0.16 | 83.90% |
| 16 | 0 | 2 | 1 | 1 | 2 | 1.79 | 0.22 | 85.49% |
| 17 | 1 | 1 | 0.5 | 0.5 | 1 | 1.77 | 0.12 | 85.21% |
| 18 | 2 | 0 | 0 | 0 | 0 | 1.37 | 0.18 | 83.64% |
| 19 | 2 | 0 | 0 | 0 | 2 | 1.40 | 0.19 | 85.12% |
| 20 | 2 | 0 | 0 | 1 | 0 | 2.13 | 0.19 | 79.15% |
| 21 | 2 | 0 | 0 | 1 | 2 | 1.95 | 0.18 | 74.60% |
| 22 | 2 | 0 | 1 | 0 | 0 | 1.64 | 0.18 | 81.93% |
| 23 | 2 | 0 | 1 | 0 | 2 | 1.42 | 0.12 | 82.77% |
| 24 | 2 | 0 | 1 | 1 | 0 | 1.99 | 0.37 | 81.37% |
| 25 | 2 | 0 | 1 | 1 | 2 | 2.19 | 0.73 | 82.96% |
| 26 | 2 | 2 | 0 | 0 | 0 | 1.91 | 0.73 | 85.85% |
| 27 | 2 | 2 | 0 | 0 | 2 | 1.82 | 0.24 | 84.68% |
| 28 | 2 | 2 | 0 | 1 | 0 | 2.10 | 0.15 | 82.18% |
| 29 | 2 | 2 | 1 | 0 | 0 | 2.69 | 0.49 | 78.89% |

Inventive compositions in Base Formula A

| | | | | | | | | |
|---|---|---|---|---|---|---|---|---|
| 30 | 2 | 2 | 0 | 1 | 2 | 3.17 | 0.68 | 80.03% |
| 31 | 2 | 2 | 1 | 0 | 2 | 4.02 | 1.02 | 77.13% |
| 32 | 2 | 2 | 1 | 1 | 0 | 3.91 | 0.24 | 78.76% |
| 33 | 2 | 2 | 1 | 1 | 2 | 3.74 | 0.44 | 75.49% |

TABLE 4
Homogenizing Power and Transparency Values for Compositions Prepared With Base Formula B

| | | | Boron | Bismuth | | Homogenizing | | |
|---|---|---|---|---|---|---|---|---|
| Formula # | NLK-506 | Aerogel | Nitride | Oxychloride | Silica | Power | Stdev | Transparency |
| 1 | 0 | 0 | 0 | 0 | 0 | 1.46 | 0.13 | 88.18% |
| Base | | | | | | | | |
| Formula B | | | | | | | | |

Test Compositions in Base Formula B

| | | | | | | | | |
|---|---|---|---|---|---|---|---|---|
| 2 | 0 | 0 | 0 | 0 | 2 | 1.65 | 0.38 | 88.01% |
| 3 | 0 | 0 | 0 | 1 | 0 | 1.67 | 0.32 | 80.29% |
| 4 | 0 | 0 | 0 | 1 | 2 | 1.99 | 0.17 | 78.13% |
| 5 | 0 | 0 | 1 | 0 | 0 | 1.93 | 0.23 | 79.36% |
| 6 | 0 | 0 | 1 | 0 | 2 | 1.56 | 0.29 | 83.44% |
| 7 | 0 | 0 | 1 | 1 | 0 | 1.72 | 0.09 | 75.79% |
| 8 | 0 | 0 | 1 | 1 | 2 | 1.21 | 0.18 | 78.05% |
| 9 | 0 | 2 | 0 | 0 | 0 | 1.15 | 0.06 | 82.72% |
| 10 | 0 | 2 | 0 | 0 | 2 | 1.41 | 0.08 | 77.83% |
| 11 | 0 | 2 | 0 | 1 | 0 | 1.54 | 0.22 | 79.09% |
| 12 | 0 | 2 | 0 | 1 | 2 | 1.56 | 0.19 | 77.56% |
| 13 | 0 | 2 | 1 | 0 | 0 | 1.28 | 0.24 | 70.95% |
| 14 | 0 | 2 | 1 | 0 | 2 | 1.5 | 0.18 | 75.14% |
| 15 | 0 | 2 | 1 | 1 | 0 | 1.4 | 0.13 | 77.24% |
| 16 | 0 | 2 | 1 | 1 | 2 | 1.64 | 0.15 | 75.39% |
| 17 | 1 | 1 | 0.5 | 0.5 | 1 | 1.41 | 0.61 | 74.43% |
| 18 | 2 | 0 | 0 | 0 | 0 | 1.82 | 1.53 | 76.99% |
| 19 | 2 | 0 | 0 | 0 | 2 | 1.72 | 1.29 | 79.49% |
| 20 | 2 | 0 | 0 | 1 | 0 | 1.11 | 0.17 | 79.49% |
| 21 | 2 | 0 | 0 | 1 | 2 | 1.21 | 0.04 | 80.12% |
| 22 | 2 | 0 | 1 | 0 | 0 | 1.45 | 0.12 | 79.49% |
| 23 | 2 | 0 | 1 | 0 | 2 | 1.72 | 0.21 | 79.75% |
| 24 | 2 | 0 | 1 | 1 | 0 | 1.45 | 0.33 | 71.32% |
| 25 | 2 | 0 | 1 | 1 | 2 | 1.71 | 0.21 | 81.63% |
| 26 | 2 | 2 | 0 | 0 | 0 | 1.69 | 0.58 | 73.00% |
| 27 | 2 | 2 | 0 | 0 | 2 | 2.47 | 1.01 | 70.69% |
| 28 | 2 | 2 | 0 | 1 | 0 | 1.53 | 0.29 | 73.06% |
| 29 | 2 | 2 | 1 | 0 | 0 | 2.05 | 1.3 | 68.17% |
| 30 | 2 | 2 | 0 | 1 | 2 | 2.61 | 0.65 | 71.11% |
| 31 | 2 | 2 | 1 | 0 | 2 | 1.71 | 0.23 | 66.09% |
| 32 | 2 | 2 | 1 | 1 | 0 | 1.92 | 0.23 | 71.03% |
| 33 | 2 | 2 | 1 | 1 | 2 | 2.16 | 0.22 | 64.97% |

TABLE 5
Homogenizing Power and Transparency Values for Comparative
Compositions Prepared With Base Formula C

| | | | Boron | Bismuth | | Homogenizing | | |
|---|---|---|---|---|---|---|---|---|
| Formula # | NLK-506 | Aerogel | Nitride | Oxychloride | Silica | Power | Stdev | Transparency |
| 1 | 0 | 0 | 0 | 0 | 0 | 1.36 | 0.06 | 87.37% |
| Base | | | | | | | | |
| Formula C | | | | | | | | |

Test Compositions in Base Formula C

| | | | | | | | | |
|---|---|---|---|---|---|---|---|---|
| 2 | 0 | 0 | 0 | 0 | 2 | 1.44 | 0.16 | 85.46% |
| 3 | 0 | 0 | 0 | 1 | 0 | 1.36 | 0.16 | 85.57% |
| 4 | 0 | 0 | 0 | 1 | 2 | 1.74 | 0.43 | 83.68% |
| 5 | 0 | 0 | 1 | 0 | 0 | 1.43 | 0.35 | 82.82% |
| 6 | 0 | 0 | 1 | 0 | 2 | 1.56 | 0.04 | 81.08% |
| 7 | 0 | 0 | 1 | 1 | 0 | 1.69 | 0.31 | 82.62% |
| 8 | 0 | 0 | 1 | 1 | 2 | 1.60 | 0.05 | 81.23% |
| 9 | 0 | 2 | 0 | 0 | 0 | 1.44 | 0.34 | 85.43% |
| 10 | 0 | 2 | 0 | 0 | 2 | 1.62 | 0.15 | 84.08% |
| 11 | 0 | 2 | 0 | 1 | 0 | 1.44 | 0.35 | 83.76% |
| 12 | 0 | 2 | 0 | 1 | 2 | 1.69 | 0.24 | 81.65% |
| 13 | 0 | 2 | 1 | 0 | 0 | 1.69 | 0.18 | 81.33% |
| 14 | 0 | 2 | 1 | 0 | 2 | 1.85 | 0.25 | 81.04% |
| 15 | 0 | 2 | 1 | 1 | 0 | 1.67 | 0.08 | 81.47% |
| 16 | 0 | 2 | 1 | 1 | 2 | 1.72 | 0.27 | 82.92% |
| 17 | 1 | 1 | 0.5 | 0.5 | 1 | 1.65 | 0.12 | 82.05% |
| 18 | 2 | 0 | 0 | 0 | 0 | 1.73 | 0.15 | 83.12% |
| 19 | 2 | 0 | 0 | 0 | 2 | 1.54 | 0.37 | 82.51% |
| 20 | 2 | 0 | 0 | 1 | 0 | 2.19 | 0.62 | 79.07% |
| 21 | 2 | 0 | 0 | 1 | 2 | 2.03 | 0.54 | 74.61% |
| 22 | 2 | 0 | 1 | 0 | 0 | 1.96 | 0.55 | 76.68% |
| 23 | 2 | 0 | 1 | 0 | 2 | 2.18 | 0.25 | 75.58% |
| 24 | 2 | 0 | 1 | 1 | 0 | 2.43 | 0.29 | 71.98% |
| 25 | 2 | 0 | 1 | 1 | 2 | 2.16 | 0.16 | 75.06% |
| 26 | 2 | 2 | 0 | 0 | 0 | 1.83 | 0.14 | 80.55% |
| 27 | 2 | 2 | 0 | 0 | 2 | 1.84 | 0.08 | 77.92% |
| 28 | 2 | 2 | 0 | 1 | 0 | 1.77 | 0.21 | 79.48% |
| 29 | 2 | 2 | 1 | 0 | 0 | 2.11 | 0.19 | 75.26% |
| 30 | 2 | 2 | 0 | 1 | 2 | 1.96 | 0.15 | 77.06% |
| 31 | 2 | 2 | 1 | 0 | 2 | 2.10 | 0.12 | 76.79% |
| 32 | 2 | 2 | 1 | 1 | 0 | 2.37 | 0.33 | 71.33% |
| 33 | 2 | 2 | 1 | 1 | 2 | 2.31 | 0.54 | 67.07% |

From the results in Table 3 above, it was surprisingly and unexpectedly found that Formulas 30, 31, 32, and 33 (inventive compositions) demonstrated the highest homogenizing power values, ranging from 3.17 to 4.02. Thus, the combination of the NLK-506 and the aerogel particles with at least two of the fillers (boron nitride, silica or bismuth oxychloride) in a composition prepared from base formula A resulted in a synergistic effect as evidenced by those formulas whose homogenizing power was greater than 2.7 (formulas 30, 31, 32, and 33), compared to those formulas or test compositions which did not have this combination of particles and fillers. This synergistic effect is related to the ability of the inventive compositions to significantly improve the evenness of color or reduce color variations on a substrate.

It was also surprisingly found that the homogenizing power values for formulas 30, 31, 32 and 33 in Table 3 were significantly higher, that is, greater than 2.7, compared to the homogenizing power values of all the formulas or test compositions in Tables 4 and 5. At the same time, the % transparency values of the inventive compositions were at least 75%.

The results also show the effect of the base formulas on the homogenizing power of the compositions above. Overall, higher homogenizing power values were achieved by the test compositions prepared with base formula A. In particular, the homogenizing power values of the inventive compositions (formulas 30, 31, 32 and 33) were higher than those of the corresponding formulas in Tables 4, and 5.

In addition, higher homogenizing power values were obtained for the inventive compositions over those of the corresponding formulas in Tables 4 and 5 even when the contribution of the base formulas themselves to the measured homogenizing power were subtracted (contributions were 0.81 for base formula A, 1.46 for base formula B and 1.36 for base formula C). At the same time, the transparency values for the inventive compositions were higher than those of the corresponding formulas in Tables 4 and 5.

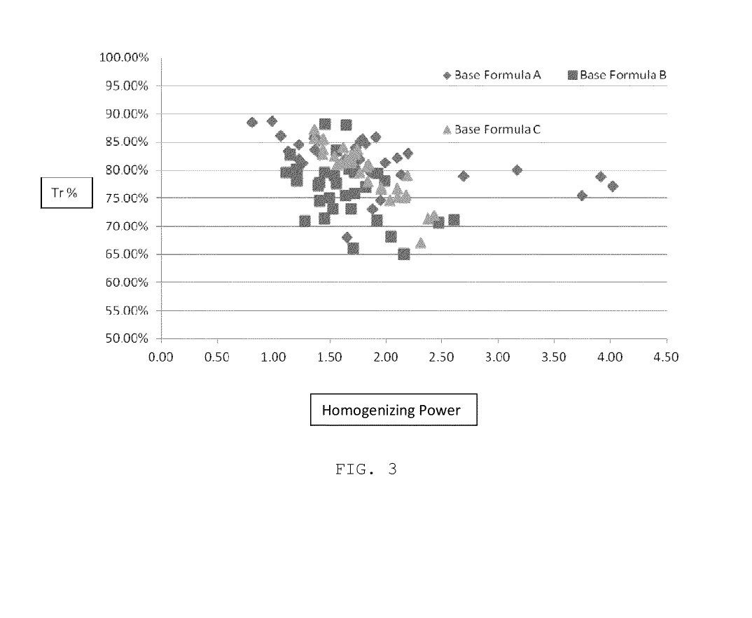

FIG. 3 represents the relationship between transparency versus homogenizing power for the test compositions prepared using three different base formulas A, B, and C (Tables 3 to 5 above). The inventive compositions prepared with base formula A, that is, formulas 30, 31, 32, and 33, demonstrated the greatest homogenizing power.

### Example 3

### In Vivo Studies

The skin parameters, visible spots, pores, lines and even skin tone, on the skin of the faces of human test volunteers were evaluated in three different ways: visual observation, chromatographic study and Visioface study.

Visual Observations by Expert Evaluators:

It was visually observed that after the application of the inventive compositions onto the facial skin of human volunteers, the visibility of skin imperfections such as pores, fine lines, dark circles around the eyes, spots or pigmentation was significantly reduced. The inventive compositions also significantly improved the evenness of the skin tone or color.

Chromatographic Study

Assessments of reduction in skin color discoloration were made via chromatographic measurements on:
bare skin (control; no product applied on skin);
skin onto which base formula A was applied; and
skin onto which the inventive compositions, formulas 31 and 32 of Table 3 (inventive compositions), were applied.

Human volunteers applied 0.25 grams of formula on their faces.

TABLE 6
Results of chromatographic study, n = 3 panelists (3 skin types)

| | | | |
|---|---|---|---|
| 1^{st} panelist | 2^{nd} panelist | 3^{rd} panelist | |
| ΔE_{ave (n=4 site)} | ΔE_{ave (n=4 site)} | ΔE_{ave (n=4 site)} | Average |

Base formula A

| | | | |
|---|---|---|---|
| −0.92 | −0.2 | −1.79 | −0.97 |

Sample 31 (inventive composition)
(Homogenizing Power - 4.09 (Max))

| | | | |
|---|---|---|---|
| −2.35 | −3.08 | −2.04 | −2.49 |

Sample 32 (inventive composition)
(Homogenizing Power - 3.91)

| | | | |
|---|---|---|---|
| −1.35 | −4.8 | −0.79 | −2.31 |

The results in Table 6 show that more negative ΔEₐᵥₑ values were obtained for the skin with the inventive compositions as compared to the skin with the placebo cream. A more negative ΔEₐᵥₑ value means that there was a greater change in the color of the skin after the inventive composition was applied onto the skin (compared to the initial color or tone of the bare skin). These results indicate that the inventive compositions imparted greater coverage and homogenizing effects or reduction of skin discoloration.

Visioface Study:

The Visioface tool (Courage-khazaka) employs a CCD camera and a software that analyzes the skin for pores, lines and eveness of skin tone or color. The Visioface tool was used to analyze the skin for the number of pores and pore size as well as for eveness of skin tone or color.

FIG. 4 represents the Visioface visual images of bare skin (control; no product applied on skin) and skin onto which Formula 31 of Table 3 above was applied (inventive composition).

An amount of 0.25 grams was applied to the face by a human volunteer.

The images show that there was an immediate effect of significant reduction in the number of pores as well as less discoloration (or more even skin tone) for the skin with the inventive composition compared to the bare skin.

### Example 4

### Stability of Inventive Compositions

The inventive composition (Base formula A+2% by weight NLK-506, 2% by weight Aerogel, 1% boron nitride and 2% silica) were shown to be stable up to 8 weeks in a controlled chamber at various temperatures at 5° C., 25° C., 37° C., and 45° C. as well as 10 days in a Freeze/Thaw cycle where the pH and viscosity had small fluctuations and there was no phase separation.

It is to be understood that the foregoing describes preferred embodiments of the invention and that modifications may be made therein without departing from the spirit or scope of the invention as set forth in the claims.

What is claimed is:

1. A composition comprising, in a cosmetically acceptable medium:
a) at least one silicone material comprising concave and/or annular particles;
b) at least one hydrophobic silica aerogel particle;
c) at least two fillers selected from the group comprising platelet type fillers and silica particles other than (b);
d) at least one fatty substance selected from the group comprising fatty alcohols and fatty acids;
e) at least one emulsifying agent selected from the group comprising oxyethylenated and/or oxypropylenated ethers of glycerol, oxyethylenated and/or oxypropylenated ethers of fatty alcohols, fatty acid esters of polyethylene glycol, fatty acid esters of oxyethylenated and/or oxypropylenated glyceryl ethers, fatty acid esters of polyols and mixtures thereof; and
f) at least one thickening agent;
wherein a), b) and c) are present in a total amount of greater than 4% by weight, based on the total weight of the composition.

2. The composition of claim 1, wherein the particles in (a) have a mean diameter of less than or equal to about 10 μm.

3. The composition of claim 1, wherein the particles in (a) are in the form of portions of hollow spheres having a transverse cross-section with the shape of a horseshoe or arch.

4. The composition of claim 1, wherein (a) comprises a crosslinked polysiloxane with a three-dimensional structure comprising or composed of units of formula (I): SiO₂ and of formula (II): R¹SiO_{1.5} in which R¹ is an organic group having a carbon atom directly bonded to the silicon atom.

5. The composition of claim 4, wherein R¹ is chosen from a C1-C4 alkyl group, a phenyl group, an epoxy group, a (meth)acryloyloxy group, an alkenyl group, a mercaptoalkyl group, an aminoalkyl group, a haloalkyl group, a glyceroxy group, an ureido group, and cyano group.

6. The composition of claim 4, wherein (a) comprises the units (I) and (II) according to a unit (I)/unit (II) molar ratio ranging from 30/70 to 50/50.

7. The composition of claim 1, wherein (a) comprises methylsilanol/silicate crosspolymer.

8. The composition of claim 1, wherein the particles in (a) are present in an amount ranging from about 0.1 to about 15% by weight, based on the total weight of the composition.

9. The composition of claim 1, wherein the hydrophobic silica aerogel particles have a specific surface area per unit of mass (SM) ranging from about 500 to about 1500 m2/g and a size expressed as the mean volume diameter (D[0.5]), ranging from about 1 to about 1500 μm.

10. The composition of claim 1, wherein (b) is silica silylate.

11. The composition of claim 1, wherein (b) is present in an amount ranging from about 0.1 to about 15% by weight, based on the total weight of the composition.

12. The composition of claim 1, wherein (c) includes a platelet type filler having a refractive index of greater than or equal to 1.6 and a particle size ranging from about 1 μm to about 30 μm.

13. The composition of claim 1, wherein the platelet type filler of (c) is selected from the group comprising boron nitride and bismuth oxychloride.

14. The composition of claim 1, wherein the silica particles of (c) comprise porous silica microspheres.

15. The composition of claim 1, wherein (c) comprises (i) a platelet type filler selected from the group comprising boron nitride and bismuth oxychloride, and (ii) silica particles comprising porous silica microspheres.

16. The composition of claim 1, wherein (c) is present in an amount ranging from about 0.1 to about 10% by weight, based on the total weight of the composition.

17. The composition of claim 1, wherein (d) is selected from the group comprising stearic acid, myristic acid, oleic acid, linoleic acid, linolenic acid, cetyl alcohol, cetearyl alcohol, isostearyl alcohol, oleyl alcohol, and mixtures thereof.

18. The composition of claim 1, wherein (e) comprises glyceryl esters and polyethylene glycol esters of fatty acids.

19. The composition of claim 1, wherein (f) is selected from the group comprising from cellulose-based thickening agents, high molecular weight glycols, polyacrylamide-based thickening agents, and mixtures thereof.

20. The composition of claim 1, wherein the composition further comprises a nonvolatile silicone oil.

21. The composition of claim 1, wherein (a), (b) and (c) are present in a total amount ranging from greater than 4% to about 10% by weight, based on the total weight of the composition.

22. The composition of claim 1, wherein the composition is an emulsion.

23. The composition of claim 1, wherein the composition is a skin care product, a make-up product, a suncare product, a concealer product, or a skin care base or make-up base or primer.

24. The composition of claim 1, wherein the composition has a homogenizing power of greater than 2.7 when applied onto skin.

25. The composition of claim 1, wherein the composition has a homogenizing effect on skin.

26. A process for reducing the visibility of imperfections or imparting a homogenizing effect on skin, comprising applying the composition of claim 1 onto the skin in an amount sufficient to reduce the visibility of imperfections or provide the homogenizing effect.

27. A cosmetic composition comprising, in a cosmetically acceptable medium:
a) from about 0.5 to about 3% by weight of a methylsilanol/silicate crosspolymer;
b) from about 0.5 to about 3% by weight of hydrophobic silica aerogel particles comprising silica silylate;
c) from about 0.5 to about 4% by weight in total of at least two fillers selected from boron nitride of a platelet type, bismuth oxychloride of a platelet type, and silica particles other than (b) comprising porous silica microspheres;
d) from about 0.25 to about 5% by weight of at least one fatty substance selected from stearic acid, myristic acid, oleic acid, linoleic acid, linolenic acid, cetyl alcohol, cetearyl alcohol, isostearyl alcohol, oleyl alcohol, and mixtures thereof;
e) from about 0.25 to about 5% by weight of at least one emulsifying agent comprising glyceryl esters, polyethylene glycol esters of fatty acids, and mixtures thereof; and
f) at least one thickening agent comprising cellulose-based thickening agents chosen from xanthan gum, carrageenan gum, guar gum, and combinations thereof;
wherein (a), (b) and (c) are present in a total amount of greater than 4% by weight,
all weights being based on the total weight of the composition.

28. A process for reducing the visibility of imperfections or imparting a homogenizing effect on skin, comprising applying the composition of claim 27 onto the skin in an amount sufficient to reduce the visibility of imperfections or provide the homogenizing effect.
